(19) United States
(12) Patent Application Publication
(10) Pub. No.: US 2011/0172427 A1
(43) Pub. Date: Jul. 14, 2011

(54) PROCESS FOR PREPARING CERTAIN CINNAMIDE COMPOUNDS

(75) Inventors: Taiju Nakamura, Kamisu (JP); Masaaki Matsuda, Tsukuba (JP); Yongbo Hu, Chestnut Hill, MA (US); Daiju Hasegawa, Tsukuba (JP); Yorihisa Hoshino, Tsukuba (JP); Kazato Inanaga, Kamisu (JP); Minetaka Isomura, Kamisu (JP); Nobuaki Sato, Tsukuba (JP); Kazuhiro Yoshizawa, Kamisu (JP); George A. Moniz, Cambridge, MA (US); Gordon D. Wilkie, North Andover, MA (US); Francis G. Fang, Andover, MA (US); Yoshihiro Nishikawa, Kamisu (JP)

(73) Assignee: EISAI R&D MANAGEMENT CO., LTD., Tokyo (JP)

(21) Appl. No.: 13/060,979

(22) PCT Filed: Aug. 26, 2009
(86) PCT No.: PCT/US09/55079

§ 371 Date: Mar. 11, 2011

(30) Foreign Application Priority Data
Aug. 27, 2008 (US) 61/092262

(51) Int. Cl. C07D 471/04; C07C 257/06; C07D 401/14; C07D 401/04

(52) U.S. Cl. 546/119; 558/6; 546/194; 546/274.1

(57) ABSTRACT

This invention relates to a new synthesis, intermediates and precursors leading to a mixture of the compounds 11 and 12 as shown below. It also relates to the resolution of the stereoisomeric mixture to provide in substantial stereochemical purity compound 12. The synthesis of the invention involves preparation of compound 7 and compound 10 as shown below and their reaction to prepare a mixture of compound 11 and compound 12.

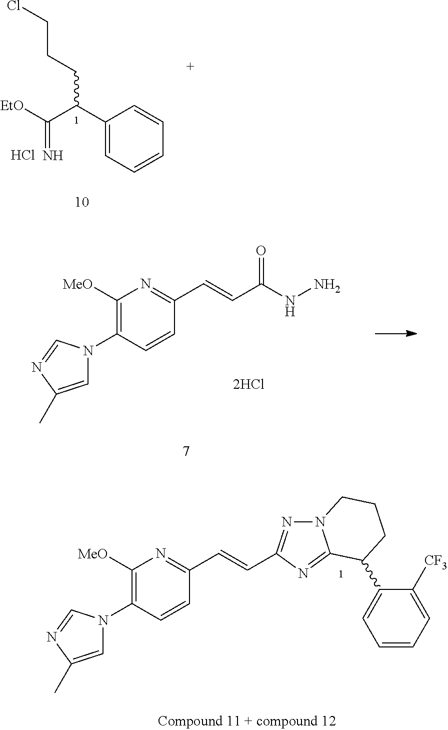

### TECHNICAL FIELD

This invention relates to a new synthesis, intermediates and precursors for preparing multicyclic cinnamide compounds.

### BACKGROUND ART

Alzheimer's disease is a disease characterized by degeneration and loss of neurons as well as formation of senile plaques and neurofibrillary degeneration. Currently, Alzheimer's disease is treated only with symptomatic treatment using a symptom improving agent typified by an acetylcholinesterase inhibitor, and a fundamental remedy to inhibit progression of the disease has not yet been developed. It is necessary to develop a method for controlling the cause of the onset of pathology in order to create a fundamental remedy for Alzheimer's Disease.

It is believed that Aβ-proteins as metabolites of amyloid precursor proteins (hereinafter referred to as APP) are highly involved in degeneration and loss of neurons and onset of symptoms of dementia. (Non-Patent Document 1 and Non-Patent Document 2) Main molecular species of Aβ-protein are Aβ40 consisting of 40 amino acids and Aβ42 with two amino acids added at the C-terminal. The Aβ40 and Aβ42 are known to have high aggregability (Non-Patent Document 3) and to be main components of senile plaques (Non-Patent Document 4 and Non-Patent Document 5). Further, it is known that the Aβ40 and Aβ42 are increased by mutation in APP and presenilin genes which is observed in familial Alzheimer's disease (Non-Patent Document 6, Non-Patent Document 7 and Non-Patent Document 8). Accordingly, a compound that reduces production of Aβ40 and Aβ42 is expected as a progression inhibitor or prophylactic agent for Alzheimer's disease.

Aβ is produced by cleaving APP by β-secretase and subsequently by γ-secretase. For this reason, attempts have been made to create γ-secretase and β-secretase inhibitors in order to reduce Aβ production. Many of these secretase inhibitors already known are, for example, peptides and peptide mimetics such as L-685,458 (Non-Patent Document 9), LY-411,575 (Non-Patent Document 10, Non-Patent Document 11 and Non-Patent Document 12) and LY-450,139 (Non-Patent Document 13, Non-Patent Document 14 and Non-Patent Document 15). Nonpeptidic compounds are, for example, MRK-560 (Non-Patent Document 16 and Non-Patent Document 17) and compounds having a plurality of aromatic rings as disclosed in Patent Document 1. Certain cinnamide compounds with potent activity to inhibit production of Aβ42 from APP have been previously disclosed in Patent Document 2. Multicyclic cinnamide compounds with potent activity to inhibit production of Aβ42 from APP have also been disclosed in Patent Document 3.

### PRIOR ART DOCUMENTS

### Patent Documents

Patent Document 1: WO 2004/110350
Patent Document 2: US 2006/0004013
Patent Document 3: WO 2007/102580

### Non-Patent Documents

Non-Patent Document 1: Klein W L, et al; Alzheimer's disease-affected brain: Presence of oligomeric Aβ ligands (ADDLs) suggests a molecular basis for reversible memory loss, Proceeding of the National Academy of Science USA, 2003, Sep., 2; 100(18), p. 10417-10422;
Non-Patent Document 2: Nitsch R M, et al; Antibodies against β-amyloid slow cognitive decline in Alzheimer's disease, Neuron, 2003, May 22; 38, p. 547-554:
Non-Patent Document 3: Jarrett J T, et al; The carboxy terminus of the β amyloid protein is critical for the seeding of amyloid formation; Implications for the pathogenesis of Alzheimers' disease, Biochemistry, 1993, 32(18), p. 4693-4697;
Non-Patent Document 4: Glenner G G, et al, Alzheimer's disease: initial report of the purification and characterization of a novel cerebrovascular amyloid protein, Biochemical and Biophysical Research Communications, 1984, May 16, 120(3), p. 885-890;
Non-Patent Document 5: Masters C L, et al, Amyloid plaque core protein in Alzheimer disease and Down syndrome, Proceeding of the National Academy of Science USA, 1985, June, 82(12), p. 4245-4249;
Non-Patent Document 6: Gouras G K, et al, Intraneuronal Aβ42 accumulation in human brain, American Journal of Pathology, 2000, January, 156(1), p. 15-20;
Non-Patent Document 7: Schemer D, et al, Secreted amyloid β-protein similar to that in the senile plaques of Alzheimer's disease is increased in vivo by the presenilin and 2 and APP mutations linked to familial Alzheimer's disease, Nature Medicine, 1996, August, 2(8), p. 864-870;
Non-Patent Document 8: Forman M S, et al, Differential effects of the swedish mutant amyloid precursor protein on β-amyloid accumulation and secretion in neurons and nonneuronal cells, The Journal of Biological Chemistry, 1997, Dec., 19, 272(51), p. 32247-32253;
Non-Patent Document 9: Shearman M S, et al, L-685, 458, an Aspartyl Protease Transition State Mimic, Is a Potent Inhibitor of Amyloid β-Protein Precursor γ-Secretase Activity, Biochemistry, 2000, Aug., 1, 39(30), p. 8698-8704;
Non-Patent Document 10: Shearman M S, et al, Catalytic Site-Directed γ-Secretase Complex Inhibitors Do Not Discriminate Pharmacologically between Notch S3 and β-APP Cleavages, Biochemistry, 2003, June, 24, 42(24), p. 7580-7586;
Non-Patent Document 11: Lanz T A, et al, Studies of Aβ pharmacodynamics in the brain, cerebrospinal fluid, and plasma in young (plaque-free) Tg2576 mice using the γ-secretase inhibitor N2-[(2S)-2-(3,5-difluorophenyl)-2-hydroxyethanoyl]-NI-[(7S)-5-methyl-6-oxo-6,7-dihydro-5H-dibenzo[b,d]azepin-7-yl]-L-alaninamide (LY-411575), The Journal of Pharmacology and Experimental Therapeutics, 2004, April, 309(1), p. 49-55;
Non-Patent Document 12: Wong G T, et al, Chronic treatment with the γ-secretase inhibitor LY-411, 575 inhibits β-amyloid peptide production and alters lymphopoiesis and intestinal cell differentiation, The Journal of Biological Chemistry, 2004, Mar., 26, 279(13), p. 12876-12882;
Non-Patent Document 13: Gitter B D, et al, Stereoselective inhibition of amyloid beta peptide secretion by LY450139, a novel functional gamma secretase inhibitor, Neurology of Aging 2004, 25, sup2, p. 571;
Non-Patent Document 14: Lanz T A, et al, Concentration-dependent modulation of amyloid-β in vivo and in vitro using the γ-secretase inhibitor, LY-450139, The Journal of Pharmacology and Experimental Therapeutics, 2006, November, 319(2) p. 924-933;
Non-Patent Document 15: Siemers E R, et al, Effects of a γ-secretase inhibitor in a randomized study of patients with Alzheimer disease, Neurology, 2006, 66, p. 602-604;
Non-Patent Document 16: Best J D, and nine others, In vivo characterization of Aβ (40) changes in brain and cerebrospinal fluid using the novel γ-secretase inhibitor N-[cis-4-[(4-chlorophenyl)sulfonyl]-4-(2,5-difluorophenyl)cyclohexyl]-1,1,1-trifluoromethanesulphonlamide (MK-560) in the rat, The Journal of Pharmacology and Experimantal Therapeutics, 2006, May 317(2) p. 786-790;
Non-Patent Document 17: Best J D, et al, The novel γ-secretase inhibitor N-[cis-4-[(4-chlorophenyl)sulfonyl]-4-(2,5-difluorophenyl)cyclo-hexyl]-1,1,1-trifluoromethanesulphonlamide (MK-560) reduces amyloid plaque deposition without evidence notch-related pathology in the Tg2576 mouse, The Journal of Pharmacology and Experimental Therapeutics, 2007, February, 320(2) p. 552-558.

### SUMMARY OF THE INVENTION

### Problem to be Solved by the Invention

As described above, a compound that inhibits production of Aβ40 and Aβ42 from APP is expected to be a therapeutic or prophylactic agent for a disease caused by Aβ which is typified by Alzheimer's disease. As reported in WO 2009/028588, compound 12 ((−)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine) is nonpeptidic compound that potently inhibits production of Aβ42 from APP. There is therefore a need to develop synthetic methods for preparing compounds such as compound 12, and their synthetic precursors, which can be used as therapeutic agents. The invention provides an improved method for synthesizing intermediates for the preparation of compounds such as compound 12, and for the preparation of substantially stereochemically pure compounds of the type of compound 12 from stereoisomeric mixtures.

### Means for Solving the Problem

Thus, the present inventions relate to the following [1] to [18]:

[1]. A process for preparing compound 12 ((−)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine) in substantial stereochemical purity, comprising the steps of:

a). forming a mixture of compound 11 and compound 12 by reacting a compound of Formula I with a compound of Formula IV as shown below:

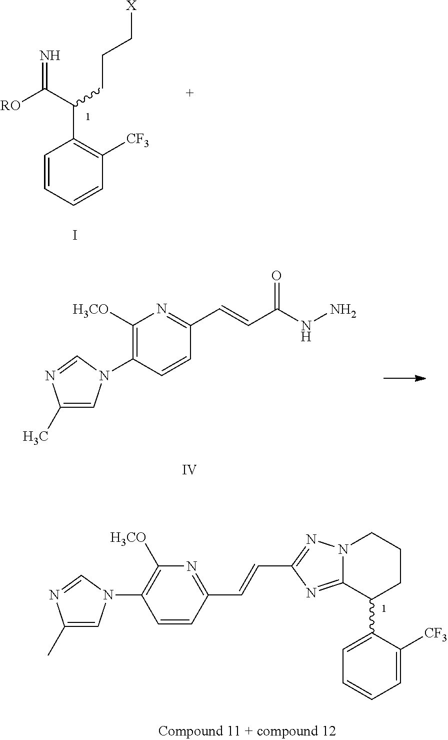

wherein X is a leaving group; R is C₁-C₆ branched or unbranched alkyl group, or C₂-C₆ branched or unbranched alkenyl group; and the stereochemistry at carbon 1 is a mixture of R and S isomers

b). forming a mixture of diastereomeric salts of compound 11 and compound 12 by treating the mixture of compound 11 and compound 12 with a chiral carboxylic acid compound;

c). crystallizing the diastereomeric salt formed of compound 12 from a solution of diastereomeric salts formed of compound 11 and compound 12; and

d). forming compound 12 from the obtained diastereomeric salt of compound 12;

[2]. A process for preparing a mixture of compound 11 and compound 12, comprising the step of reacting a compound of Formula I or a salt thereof with a compound of Formula IV or a salt thereof as shown below:

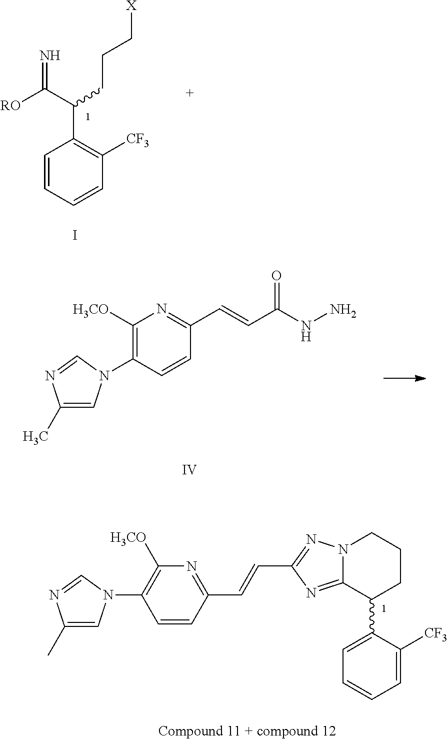

wherein X, R and the stereochemistry at carbon 1 are as defined in [1] above;
[3]. The process according to [1] or [2] above wherein the reaction is carried out in methanol, tetrahydrofuran or a mixture thereof in the presence of imidazole or sodium acetate, optionally followed by the addition of triethylamine;
[4]. A process for preparing compound 12 ((−)-2-{(E)-2-[6-methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine) in substantial stereochemical purity, comprising the steps of

a). forming a mixture of diastereomeric salts of compound 11 ((+)-2-{(E)-2-[6-methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine) and compound 12 by treating a mixture of compound 11 and compound 12 with a chiral carboxylic acid compound;

b). crystallizing the diastereomeric salt formed of compound 12 from a solution of diastereomeric salts formed of compound 11 and compound 12; and

c). forming compound 12 from the obtained diastereomeric salt of compound 12;

[5]. The process according to any one of [1], [3] and [4] above, wherein the chiral carboxylic acid compound is selected from D-dibenzoyl tartaric acid (D-DBTA), D-dipivaloyl tartaric acid (D-DPTA) and (+)-N-(1-Phenylethyl)phthalamic acid ((+)-PEPA);
[6]. The process according to any one of [1], [3], [4] and [5] above, wherein the solvent is a co-solvent mixture of 2-propanol and acetonitrile;
[7]. The process according to any one of [1], [3], [4] and [5] above, wherein the solvent is a co-solvent mixture of methanol and acetonitrile;
[8]. The process according to any one of [1], [3], [4], [5], [6] and [7] above, further comprising a second crystallization of the diastereomeric salt of compound 12 from a solvent prior to forming compound 12;
[9]. The process according to [8] above, wherein the solvent for the second crystallization is a co-solvent of 2-propanol and acetonitrile;
[10]. A D-DBTA salt of Compound 12;
[11]. A D-DPTA salt of Compound 12;
[12]. A (+)-N-(1-Phenylethyl)phthalamic acid ((+)-PEPA) salt of Compound 12;
[13]. A compound of Formula I:

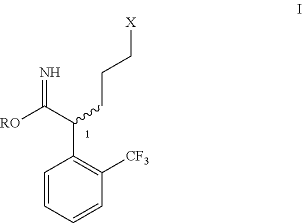

wherein X, R and the stereochemistry at carbon 1 are as defined in [1] above, or a salt thereof;
[14]. A compound of Formula III:

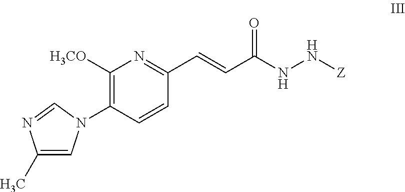

wherein Z is a hydrogen atom or a nitrogen protecting group, or a salt thereof;
[15]. The compound of Formula III or a salt thereof according to [14] above, wherein Z is a hydrogen atom;
[16]. A process for preparing a compound of Formula I, comprising the steps of

a). forming a compound of Formula VI by reacting 2-(trifluoromethyl)phenylacetonitrile with a compound of X(CH₂)₃XI as shown below:

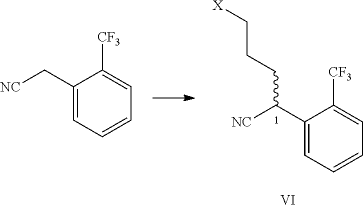

wherein X and X1 are leaving groups;

b). forming a compound of Formula I, by reacting a compound of Formula VI with ROH in the presence of an acid as shown below:

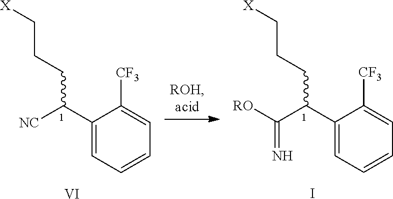

wherein X, R and the stereochemistry at carbon 1 are as defined in [1] above;
[17]. The process of [16] above, wherein the acid is in situ prepared by reacting a lower alkanoyl halide, thionyl chloride or trimethylsilyl halide with ROH;
[18]. A process for preparing a compound of Formula IV or a salt thereof, comprising the steps of

a). forming a compound of Formula III or a salt thereof by reacting N′-protected acrylohydrazide 5 or a salt thereof with a compound II or a salt thereof in the presence of palladium catalyst, a substituted phosphine of PR¹₃ and a base as shown below:

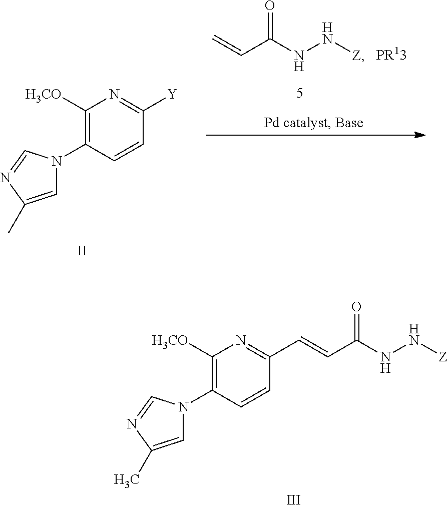

wherein Y is a leaving group; and R¹ is C₁-C₆ branched or unbranched alkyl group, or optionally substituted phenyl group;

b). forming a compound of Formula IV or a salt thereof by removing the protecting group of compound of Formula III as shown below:

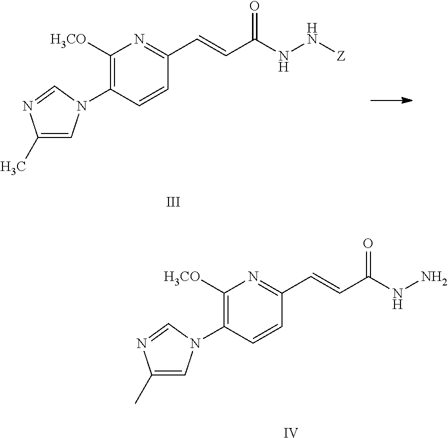

[19]. The process of [18] above, wherein dihydrochloride salt of compound of Formula IV is formed by reacting a compound of Formula III with HCl in 1-propanol;
[20]. A compound of Formula II:

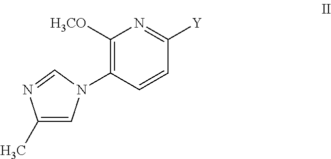

wherein Y is as defined in [18] above, or a salt thereof;
and
[21] The compound according to [20] above, wherein Y is a bromine atom.

### DETAILED DESCRIPTION OF THE INVENTION

Throughout the specification and claims, the following definitions apply:

As used herein, the term “solvent” encompasses both single solvents and co-solvent mixtures of more than one solvent.

“Alkyl” refers to a saturated straight or branched chain hydrocarbon radical. Examples include without limitation methyl, ethyl, propyl, iso-propyl, butyl, iso-butyl, tert-butyl, n-pentyl and n-hexyl.

“Alkenyl” refers to an unsaturated straight or branched chain hydrocarbon radical comprising at least one carbon to carbon double bond. Examples include without limitation ethenyl, propenyl, iso-propenyl, butenyl, iso-butenyl, tert-butenyl, n-pentenyl and n-hexenyl.

“Halo” refers to one or more of a fluoro, chloro, bromo or iodo radical.

“Leaving group” refers to halo, C₁₋₆alkylsulfonate such as methanesulfonate, or C₆₋₁₄ arylsulfonate such as p-toluenesulfonate.

“Salt thereof” refers to hydrohalide such as hydrofluoride, hydrochloride, hydrobromide and hydroiodide; inorganic acid salt such as sulfate, nitrate, perchlorate, phosphate, carbonate and bicarbonate; organic carboxylate such as acetate, oxalate, maleate, tartrate, fumarate and citrate; organic sulfonate such as methanesulfonate, trifluoromethanesulfonate, ethanesulfonate, benzenesulfonate, p-toluenesulfonate and camphorsulfonate; amino acid salt such as aspartate and glutamate; and quaternary amine.

“Isomers” refers to compounds having the same number and kind of atoms and hence the same molecular weight, but differing with respect to the arrangement or configuration of the atoms.

“Stereoisomers” refers to isomers that differ only in the arrangement of the atoms in space.

“Diastereoisomers” refers to stereoisomers that are not mirror images of each other.

“Enantiomers” refers to stereoisomers that are non-superimposable mirror images of one another. Enantiomers include “enantiomerically pure” isomers that comprise substantially a single enantiomer, for example, greater than or equal to 90%, 92%, 95%, 98%, or 99%, or equal to 100% of a single enantiomer.

“R” and “S” as terms describing isomers are descriptors of the stereochemical configuration at an asymmetrically substituted carbon atom. The designation of an asymmetrically substituted carbon atom as “R” or “S” is done by application of the Cahn-Ingold-Prelog priority rules, as are well known to those skilled in the art, and described in the International Union of Pure and Applied Chemistry (IUPAC) Rules for the Nomenclature of Organic Chemistry. Section E, Stereochemistry.

An enantiomer can be characterized by the direction in which it rotates the plane of plane polarized light, as is well known to those in the chemical arts. If it rotates the light clockwise (as seen by a viewer towards whom the light is traveling), that enantiomer is labeled (+), and is denoted dextrorotatory. Its mirror-image will rotate plane polarized light in a counterclockwise direction, and is labeled (−), or levorotatory. The direction of rotation of plane polarized light by an enantiomerically pure compound, termed the sign of optical rotation, may be readily measured in standard device known as a polarimeter.

“Racemic” refers to a mixture containing equal parts of individual enantiomers.

“Non-racemic” refers to a mixture containing unequal parts of individual enantiomers. A non-racemic mixture may be enriched in the R- or S-configuration, including, without limitation, about 50/50, about 60/40, and about 70/30 R- to S-enantiomer, or S- to R-enantiomer, mixtures.

“Substantially stereochemically pure” and “substantial stereochemical purity” refer to enantiomers or diastereomers that are in enantiomeric excess or diastereomeric excess, respectively, equal to or greater than 80%. In some embodiments, “Substantially stereochemically pure” and “substantial stereochemical purity” refer to enantiomers or diastereomers that are in enantiomeric excess or diastereomeric excess, respectively, equal to or greater than 87%, equal to or greater than 90%, equal to or greater than 95%, equal to or greater than 96%, equal to or greater than 97%, equal to or greater than 98%, or equal to or greater than 99%.

“Enantiomeric excess” (ee) of an enantiomer is [(the mole fraction of the major enantiomer) minus the (mole fraction of the minor enantiomer)]×100. Diastereomeric excess (de) of a diastereomer in a mixture of two diastereomers is defined analogously.

This invention relates to a new synthesis, intermediates and precursors leading to substantially stereochemically pure compound 12. One embodiment of the invention is depicted in Scheme I.

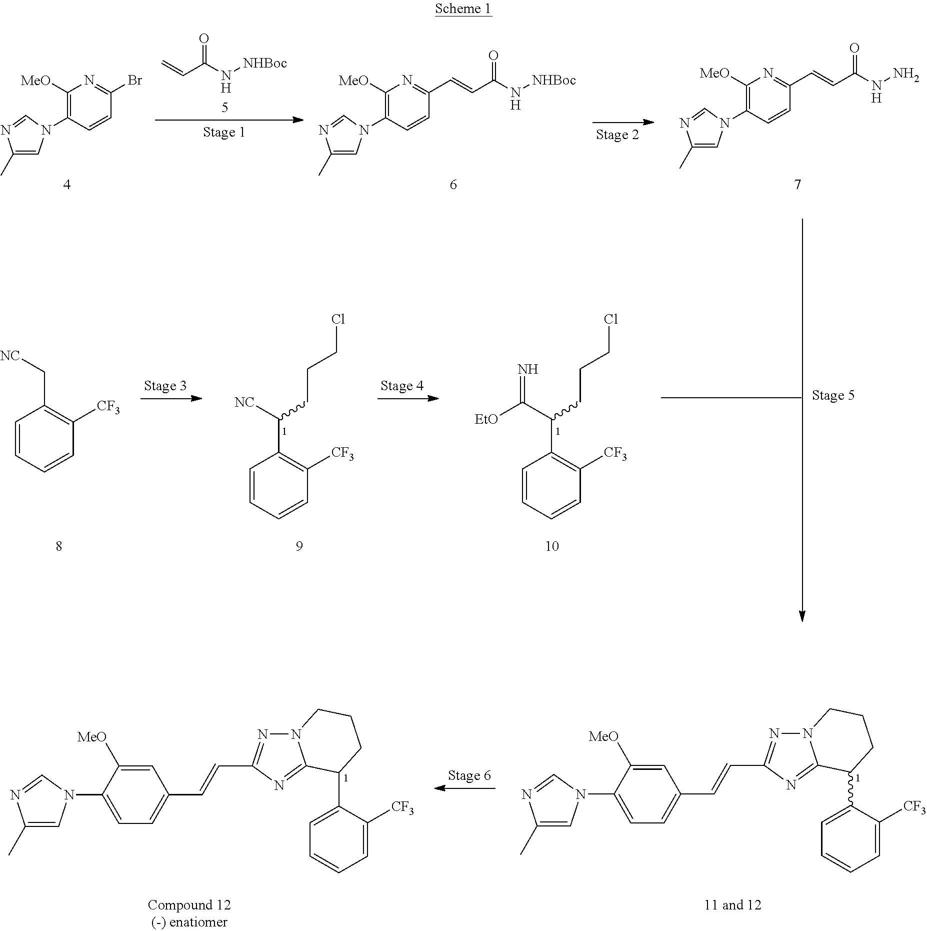

Compounds 11 and 12 have an asymmetrically substituted carbon atom, noted by a numeral 1 in Scheme 1. Certain of the intermediate compounds described herein also have an asymmetrically substituted carbon atom, which is noted by a numeral 1 in the Schemes and Formulae. The synthesis of the invention begins with the synthesis of compound 10 from compound 9, and compound 7 from compound 4 via compound 6, as depicted in Scheme 1. Compound 10 and compound 7 are then reacted together to form a mixture of stereoisomers comprising compounds 11 and 12. Substantially stereochemically pure compound 12, is obtained by preparation of the D-dibenzoyl tartaric acid (D-DBTA) salt, the D-dipivaloyl tartaric acid (D-DPTA) salt, or the (+)-N-(1-Phenylethyl)phthalamic acid ((+)-PEPA) salt of the stereoisomeric mixture followed by crystallization to afford compound 12 as the (−)-enantiomer, that is levorotatory with respect to the rotation of the plane of polarized light. Compounds 4, 6, 7 and 10 represent separate embodiments of the invention.

In Scheme 1, all of compounds 4, 6 to 12 may be in the form of a salt thereof.

One embodiment of the invention is a compound of Formula I:

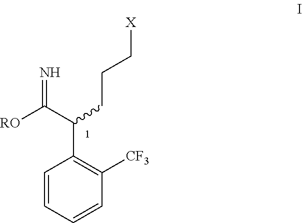

or a salt thereof,
wherein X is a leaving group; R is C₁-C₆ branched or unbranched alkyl, or C₂-C₆ branched or unbranched alkenyl; and the stereochemistry at carbon 1 is R, S, or a mixture of R and S isomers. In some embodiments, X is a leaving group chosen from halo, C₁₋₆alkylsulfonate, or C₆₋₁₄ arylsulfonate. In some embodiments, X is a leaving group chosen from halo, mesylate, or tosylate. In some embodiments, X is halo chosen from chloro, bromo, and iodo. In some embodiments, R is C₂-C₄ branched or unbranched alkyl. In some embodiments, R is C₁-C₃ branched or unbranded alkyl. In some embodiments, R is C₃-C₅ branched or unbranched alkyl. In some embodiments, R is C₄-C₆ branched or unbranched alkyl. In some embodiments, R is ethyl. Imidate compound 10 in Scheme 1 is one embodiment of compounds of Formula I (X═Cl and R=ethyl).

Another embodiment of the invention is a compound of Formula II:

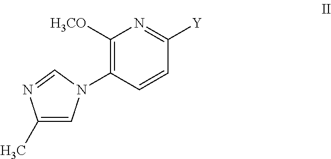

or a salt thereof,
wherein Y is a leaving group, preferably halo or triflate. In some embodiments, Y is halo selected from bromo or iodo. Bromo compound 4 in Scheme 1 is a compound of Formula II.

Another embodiment of the invention is a compound of Formula III:

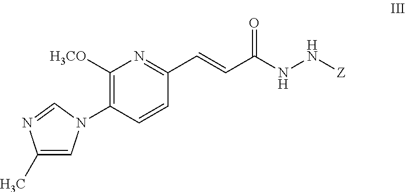

or a salt thereof,
wherein Z is a hydrogen atom or a nitrogen-protecting group. The nitrogen-protecting group used varies according to the starting material and is not specifically limited insofar as the group does not inhibit the production of a compound of Formula III and it can be removed without affect the other functional groups of a compound of Formula III. Examples of a nitrogen-protecting group include a benzyloxycarbonyl (Cbz) group, a methoxycarbonyl group, an ethoxycarbonyl group, a tert-butoxycarbonyl group (tBoc), a 9-fluorenylmethyloxycarbonyl group (Fmoc) and trichloroethyloxycarbonyl group (Troc). In one embodiment, substituted pyridine compound 6 in Scheme 1 is a compound of Formula III, wherein Z is tert-butoxycarbonyl group.

Another embodiment of the invention is a compound of Formula IV:

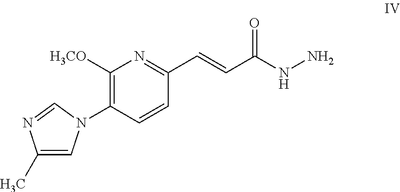

or a salt thereof. Compound 7 in Scheme 1 is a compound of Formula IV. A compound of Formula IV is one embodiment of compounds of Formula III (Z═H).

Another embodiment of the invention is process for preparing compounds of Formula V, comprising the step of reacting a compound of Formula I with a compound of Formula IV as shown in Scheme 2.

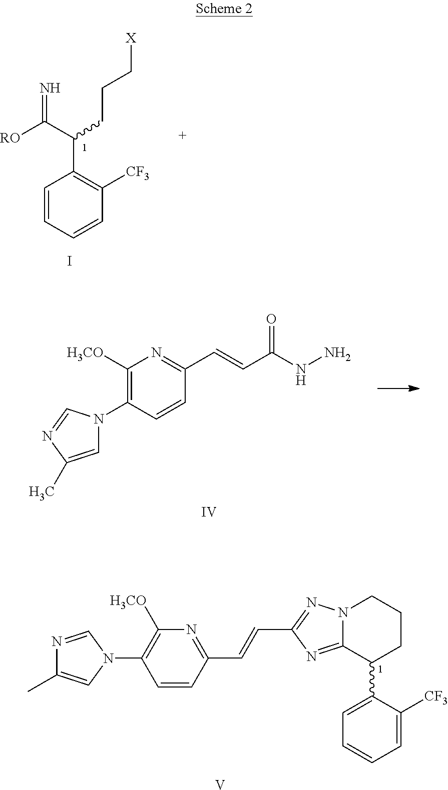

In some embodiment, the reaction takes place in methanol in the presence of imidazole.

In Scheme 2, compounds I and IV may be in the form of a salt thereof.

Another embodiment of the invention is a process for resolving compound V into its two enantiomers, compound 11 and compound 12, by treating a mixture of compound 11 and compound 12 with a chiral carboxylic acid compound, followed by crystallizing one of the diastereomeric salt selectively.

Another embodiment of the invention is the preparation of compound 12, the (−)-enantiomer of Formula V, by selective crystallization from a solution of the D-DBTA salts of compound 11 and compound 12. Compound 11 is the dextrorotatory (positive sign of optical rotation) enantiomer of Formula V, and compound 12 is the levorotatory (negative sign of optical rotation) enantiomer of Formula V.

In some embodiment, a chiral carboxylic acid compound used is D-dibenzoyltartaric acid (D-DBTA), tartaric acid (D-DPTA) or (+)-N-(1-Phenylethyl)phthalamic acid ((+)-PEPA).

Another embodiment of the invention is a salts of compound 12 with a chiral carboxylic acid compound.

In some embodiment, the salt is a D-dibenzoyltartaric acid (D-DBTA) salt, D-dipivaloyl tartaric acid (D-DPTA) salt or (+)-N-(1-Phenylethyl)phthalamic acid ((+)-PEPA) salt of compound 12 as shown in Scheme 3.

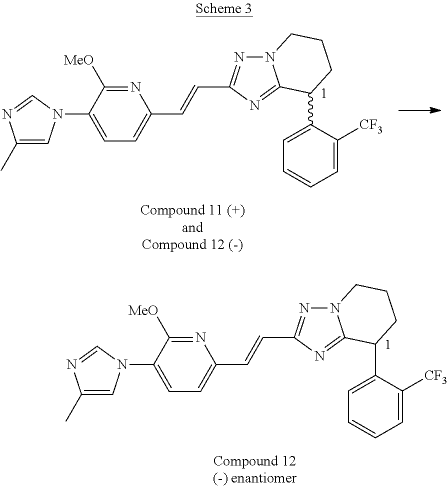

Scheme 4 depicts a synthetic route whereby the compounds 11 and 12 may be prepared as a mixture of stereoisomers and then separated by chromatography on a chiral column. This process may be used to obtain seed crystals of compounds 11 and 12 commonly used in the process of Scheme 4 and the process of Scheme 1.

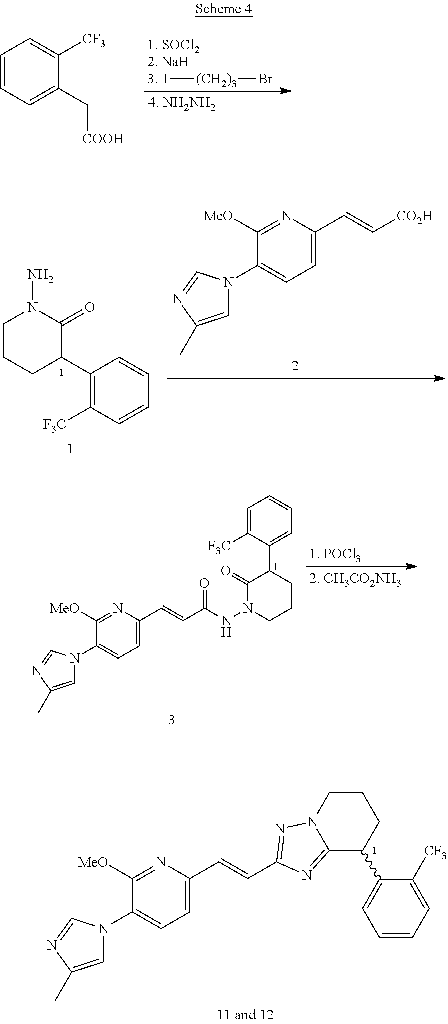

### Preparation of Imidates of Formula I

Imidates of Formula I can be prepared by reacting nitrile compounds VI with a lower alcohol of ROH, such as methanol, ethanol and 1-propanol in the presence of acid, for example gaseous HCl, as shown in Scheme 5,

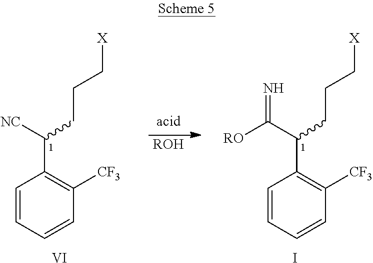

This process can be performed according to a method described in J. Am. Chem. Soc., 1990, Vol. 112, pp. 6672-6679, for example. The reaction can be performed with or without solvent. And there is no particular restriction on the solvent used in the reaction as long as it dissolves the starting material to some extent and does not inhibit the reaction, which may be any of an organic solvent, but preferred examples of the solvent include a solvent such as benzene, toluene, xylene, methanol, ethanol, 1-propanol, isopropanol, ethyl acetate, tetrahydrofuran, ether, 1,4-dioxane, 1,2-dimethoxyethane, dichloromethane, 1,2-dichloroethane or a mixture thereof, and more preferable examples thereof include a solvent such as toluene, methanol, ethanol, 1-propanol, isopropanol or ethyl acetate.

There is no particular restriction on the acid used in the reaction as long as it does not inhibit the reaction and it does not cause undesirable side reaction, but preferred examples of the acid include hydrogen halide such as HCl or HBr, and more preferable examples thereof is gaseous HCl.

This process can also be performed according to a method described in Eur. J. Org. Chem., 2005, pp. 452-456, for example. The procedures include in situ generation of the acid by adding lower alkanoyl halide to a mixture of nitrite compound VI and lower alcohol. Since this procedure does not use gaseous hydrogen halide, it is simple and easy to scale up the reaction. And the Imidate I can be isolated from the reaction mixture easily. Instead of lower alkanoyl halide, thionyl halide such as thionyl chloride or trimethylsilyl halide such as trimethylsilyl chloride may be used.

The amount of the lower alcohol used in the reaction may be increased or decreased accordingly, but the amount thereof is preferably, for example, a 3.0-fold to 24-fold molar amount, and more preferably, for example, a 5.0-fold to 20-fold molar amount relative to nitrile compound VI.

The amount of the acid used in the reaction may be increased or decreased accordingly, but the amount thereof is preferably, for example, a 2.0-fold to 20-fold molar amount, and more preferably, for example, a 4.0-fold to 16-fold molar amount relative to nitrile compound VI.

The ratio of the lower alcohol to the acid may be increased or decreased accordingly as long as the amount of the alcohol is excess to that of the acids and the excess amount of the alcohol is equimolar or an excess to one mole of nitrile compound VI. The preferred ratio thereof is between about 1.2:1 to about 1.5:1.

The reaction temperature generally varies depending on the starting material, the solvent and the reagent used in the reaction, and can be changed accordingly. The reaction temperature is preferably, for example, from −10° C. to 30° C., and more preferably, for example, from 0° C. to 10° C.

The reaction time generally varies depending on the starting material, the solvent and the reagent used in the reaction as well as the reaction temperature and the progress of the reaction, and can be increased or decreased accordingly. After addition of the acid, the reaction is generally completed in preferably, for example, 4 to 120 hours, and more preferably, for example, from 12 to 72 hours at the above reaction temperature.

Nitrile compound VI is prepared by reacting 2-(trifluoromethyl)phenylacetonitrile with a compound of X(CH₂)₃X1 as shown below:

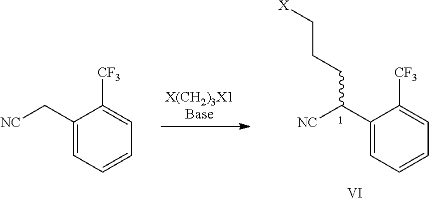

wherein X and X1 are a leaving group.

Nitrile compound 9 in Scheme 1 is one embodiment of compounds of Formula VI (X═Cl). This process can be performed according to a method described in 3. Med. Chem., 1999, Vol. 42, pp. 4680-4694, for example.

There is no particular restriction on the solvent used in the reaction as long as it dissolves the starting material to some extent and does not inhibit the reaction, which may be any of an organic solvent, but preferred examples of the solvent include a solvent such as toluene, xylene, tetrahydrofuran, ether, 1,2-dimethoxyethane, N,N-dimethylformamide (DMF), or a mixture thereof, and more preferable examples thereof include a solvent such as tetrahydrofuran, ether or 1,2-dimethoxyethane.

There is no particular restriction on the base used in the reaction as long as it does not inhibit the reaction and it does not cause undesirable side reaction, but preferred examples of the base include a base such as sodium hydride, potassium tert-butoxide, sodium amide, lithium diisopropylamide, lithium hexamethyldisilazide or butyllithium.

There is no particular restriction on the a compound of X(CH₂)₃X1 used in the reaction as long as it does not inhibit the reaction and it does not cause undesirable side reaction, but preferred examples include a compound such as 1-Bromo-3-chloropropane, 1-Chloro-3-iodopropane, 3-chloropropyl methanesulfonate, or 3-chloropropyl p-toluenesulfonate.

The amount of the base used in the reaction may be increased or decreased accordingly, but the amount thereof is preferably, for example, a 0.9-fold to 1.8-fold molar amount, and more preferably, for example, a 1.0-fold to 1.5-fold molar amount relative to 2-(trifluoromethyl)phenylacetonitrile.

The amount of the compound of X(CH₂)₃X1 used in the reaction may be increased or decreased accordingly, but the amount thereof is preferably, for example, a 1.0-fold to 4.0-fold molar amount, and more preferably, for example, a 1.0-fold to 2.0-fold molar amount relative to 2-(trifluoromethyl)phenylacetonitrile.

The ratio of the base to the compound of X(CH₂)₃X1 may be increased or decreased accordingly as long as the amount of the compound of X(CH₂)₃X1 is equimolar or an excess to that of the base. The preferred ratio thereof is between about 1:1 to about 1:1.5.

The reaction temperature generally varies depending on the starting material, the solvent and the reagent used in the reaction, and can be changed accordingly. The reaction temperature is preferably, for example, from −90° C. to 30° C., and more preferably, for example, from −78° C. to 10° C.

The reaction time generally varies depending on the solvent and the reagent used in the reaction as well as the reaction temperature and the progress of the reaction, and can be increased or decreased accordingly. Stirring time after addition of the base is preferably from 5 minute to 4 hours at the above reaction temperature. Then the compound of X(CH₂)₃X1 is added. Stirring time after addition of the compound of X(CH₂)₃X1 is preferably, for example, from 10 minute to 12 hours, and more preferably, for example, from 30 minutes to 4 hours at the above reaction temperature.

Alternatively, imidates of Formula I may be prepared from 2-trifluoromethyl phenylacetic acid as depicted in Scheme 5a.

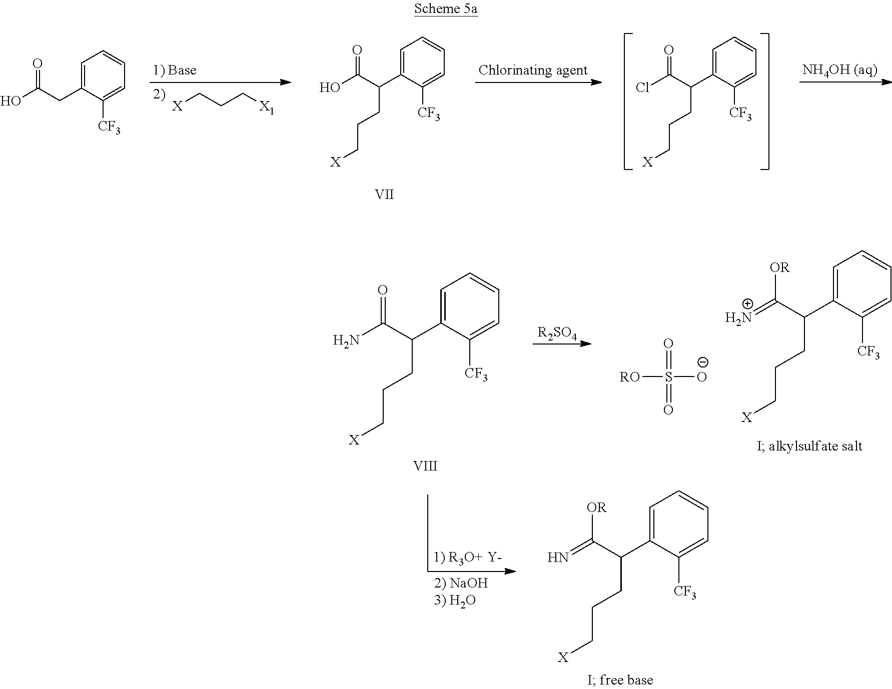

Substituted phenylacetic acid VII is prepared by making the dianion of 2-trifluoromethyl phenylacetic acid and reacting with a compound of X(CH₂)₃X1 as shown in Scheme 5a.

Substituted phenylacetic acid VII may be converted to amide VIII by reacting acid VII with a suitable chlorinating agent to convert the carboxylic acid group to the corresponding acid chloride, followed by reaction with aqueous ammonium hydroxide.

Amide VIII may be reacted with dialkylsulfates to provide imidates of Formula I as the alkylsulfate salts, as shown in Scheme 5a. Alternatively, amide VIII may be reacted with trialkyloxonium salts followed by sodium hydroxide to provide imidates of Formula I as the free bases.

### Preparation of Pyridines of Formula II

Pyridines of Formula II may be prepared by the reaction of appropriately substituted 3-(2-oxopropylformamide)pyridines or salts thereof with ammonia or an ammonium salt such as ammonium acetate in glacial acetic acid, as shown in Scheme 6

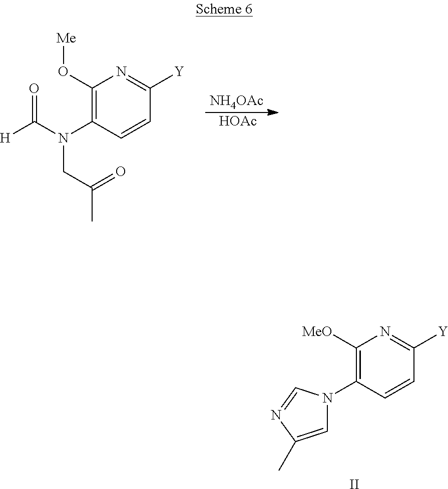

The reaction can be performed with or without solvent. And there is no particular restriction on the solvent used in the reaction as long as it dissolves the starting material to some extent and does not inhibit the reaction, which may be any of an organic solvent, but preferred examples of the solvent include a solvent such as toluene, xylene, acetic acid, tetrahydrofuran, 1,4-dioxane, formamide, acetamide, 1-methyl-2-pyrrolidone or a mixture thereof and more preferable examples include a solvent such as acetic acid or formamide.

There is no particular restriction on the ammonium salt used in the reaction as long as it does not inhibit the reaction and it does not cause undesirable side reaction, but preferred examples of the salt include an ammonium salt such as ammonium acetate or ammonium formate.

The amount of the ammonium salt used in the reaction may be increased or decreased accordingly, but the amount thereof is preferably, for example, a 3.0-fold to 20-fold molar amount, and more preferably, for example, a 5.0-fold to 10-fold molar amount relative to the substituted pyridine.

In preferred embodiment, this reaction is carried out with a 5.0-fold to 10-fold molar amount of ammonium acetate and a 10-fold to 20-fold molar amount of acetic acid. In one embodiment, the substituted pyridine is N-(6-bromo-2-methoxypyridin-3-yl)-N-(2-oxopropyl)formamide.

### Preparation of Protected Pyridyl Hydrazinecarboxylates III

The synthesis of compound 6 and similar compounds involves reaction of a substituted pyridine of Formula II or a salt thereof with a nitrogen-protected acryloylhydrazinecar-boxylate or a salt thereof to provide protected pyridyl hydrazinecarboxylates of Formula III under suitable reaction conditions. This is shown in Scheme 7.

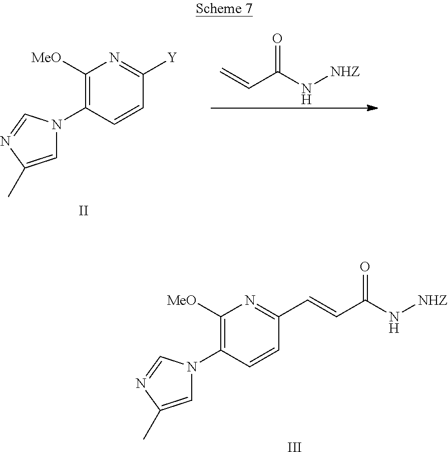

In Scheme 7, the nitrogen-protecting group Z used varies according to the starting material and is not specifically limited insofar as the group does not inhibit the production of a compound of Formula III and it can be removed without affect the other functional groups of a compound of Formula III.

The selection, incorporation of, and removal, of nitrogen protecting groups as above is well known to those in the chemical arts. [P. G. M. Wuts and T. H. Greene, Greene's Protective Groups in Organic Synthesis, 4^{th} Edition, John Wiley & Sons 2007, Chapter 7.] Preferred examples of the nitrogen-protecting group include a nitrogen-protecting group such as a benzyloxycarbonyl (Cbz) group, a methoxycarbonyl group, an ethoxycarbonyl group, a tert-butoxycarbonyl group (tBoc), a 9-fluorenylmethyloxycarbonyl group (Fmoc) or trichloroethyloxycarbonyl group (Troc). In a more preferred embodiment Z is tert-butoxycarbonyl (tBoc).

Y in Formula II is a leaving group, and preferably bromo or trifluoromethanesulfonyl (triflate), with bromo being especially preferred. The reaction in Scheme 7 may be effected by reaction with palladium catalyst in the presence of a substituted phosphine and a base. Preferred examples of the palladium catalyst include a catalyst such as palladium (II) acetate (Pd(OAc)₂) or Tris(dibenzylideneacetone)dipalladium(0) Pd₂(dba)₃. In a more preferred embodiment the palladium catalyst is palladium (II) acetate.

Preferred examples of the phosphine include a phosphine such as tris(o-tolyl)phosphine or triphenylphosphine. In a more preferred embodiment the phosphine is tris(o-tolyl)phosphine.

Both an organic base and an inorganic base can be used in the reaction. Preferred example of the base include a base such as diisoprpylethylamine, triethylamine or potassium carbonate. In a more preferred embodiment the base is diisopropylethylamine.

There is no particular restriction on the solvent used in the reaction as long as it dissolves the starting material to some extent and does not inhibit the reaction, which may be either an organic solvent or a water-containing solvent, but preferred examples of the solvent include a solvent such as toluene, xylene, ethanol, 1-propanol, ethyl acetate, tetrahydrofuran, 1,4-dioxane, N,N-dimethylformamide (DMF), 1-methyl-2-pyrrolidone, acetonitrile, water or a mixture of the solvent as above. In a more preferred embodiment the solvent is N,N-dimethylformamide.

The ratio of the palladium catalyst to the phosphine may be increased or decreased accordingly as long as the amount of the phosphine is equimolar or an excess to that of the palladium. The preferred ratio thereof is between about 1:1 to about 1:4, and more preferable ratio is about 1:2.

The reaction temperature generally varies depending on the starting material, the solvent and the reagent used in the reaction, and can be changed accordingly. The reaction temperature is preferably, for example, from 50° C. to 120° C., and more preferably, for example, from 90° C. to 110° C.

The product of the reaction can be isolated by crystallization without extraction.

### Preparation of Hydrazides of Formula IV

Hydrazide compound IV may be prepared from a nitrogen-protected compound of Formula III or a salt thereof by subjecting the compound of Formula III or a salt thereof to the appropriate deprotection conditions, This is shown in Scheme 8.

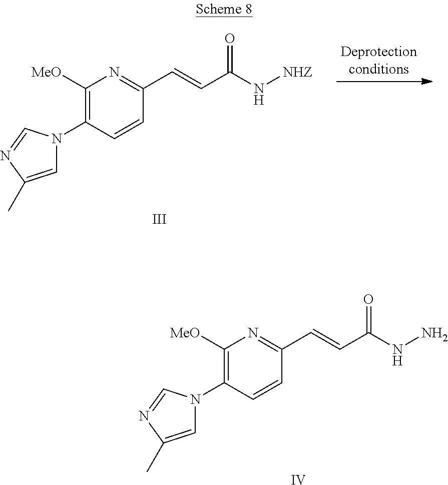

Such deprotection conditions depend on the specific protecting group, and are well known to those skilled in the art of organic synthesis. Representative procedures for removal of nitrogen-protecting groups may be found for example in Greene, 4^{th} Edition, Chapter 7.

For example a benzyloxycarbonyl (Cbz) group, a methoxycarbonyl group and an ethoxycarbonyl group can be removed under basic hydrolysis with alkali metal hydroxide such as lithium hydroxide, sodium hydroxide or potassium hydroxide. A 9-fluorenylmethyloxycarbonyl group (Fmoc) can be removed by the treatment with several secondary amines and a trichloroethyloxycarbonyl group (Troc) can be removed by using zinc.

In preferred embodiment, a tert-butoxycarbonyl group (tBoc) can be used as a protecting group and can be removed in the presence of an acid. There is no particular restriction on the acid used, but preferred examples of the acids include an acid such as hydrochloric acid, hydrobromic acid, sulfuric acid or trifluoroacetic acid. In a more preferred embodiment, deprotection conditions include treatment with hydrochloric acid in alcoholic solvent.

There is no particular restriction on the solvent used in the reaction as long as it dissolves the starting material to some extent and does not inhibit the reaction, which may be either an organic solvent or a water-containing solvent, but preferred examples of the solvent include a solvent such as toluene, xylene, ethanol, 1-propanol, isopropanol, 1-butanol, ethyl acetate, tetrahydrofuran, 1,4-dioxane, N,N-dimethylformamide (DMF), acetonitrile, water and a mixture of the solvent as above. In a more preferred embodiment the solvent is 1-propanol.

The ratio of the acid to the starting material may be increased or decreased accordingly as long as the amount of the acid is an excess to that of the starting material. The preferred ratio thereof is between about 5:1 to about 20:1, and more preferable ratio is between about 10:1 to about 15:1.

The reaction temperature generally varies depending on the starting material, the solvent and the reagent used in the reaction, and can be changed accordingly. The reaction temperature is preferably, for example, from 10° C. to 60° C., and more preferably, for example, from 40° C. to 50° C.

In particularly preferred embodiment, the procedure includes addition of the starting material to a mixture of cone hydrochloric acid and 1-propanol and separation of the product by collecting the formed crystal.

### Preparation of Compounds of Formula V

Compound 11 and compound 12 may be prepared by reacting a compound of Formula I with a compound of Formula IV under suitable reaction conditions as shown in Scheme 9.

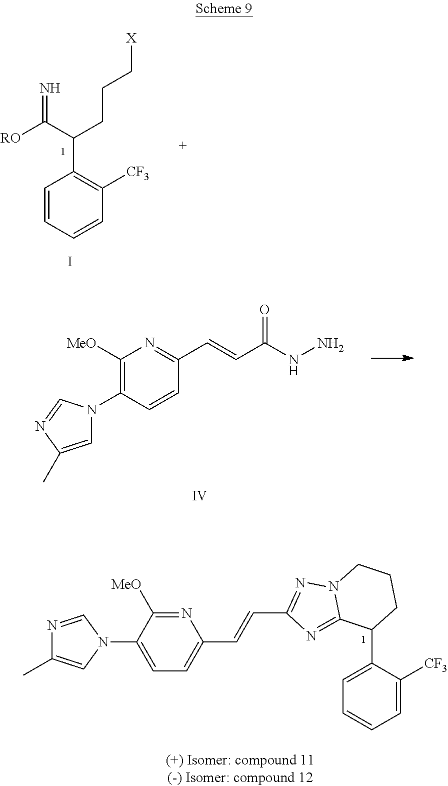

The reaction can be carried out in the presence of a base. There is no particular restriction on the base used, but preferred examples of the base include an organic base such as diisoprpylethylamine, triethylamine, pyridine, collidine or imidazole, and an inorganic base such as potassium carbonate, ammonium acetate or sodium acetate. In a preferred embodiment the base includes imidazole; sodium acetate; a mixture of imidazole and triethylamine and a mixture of sodium acetate and triethylamine.

There is no particular restriction on the solvent used in the reaction as long as it dissolves the starting material to some extent and does not inhibit the reaction, which may be either an organic solvent or a water-containing solvent, but preferred examples of the solvent include a solvent such as toluene, xylene, methanol, ethanol, 1-propanol, isopropanol, ethyl acetate, tetrahydrofuran, 1,4-dioxane, N,N-dimethylformamide (DMF), acetonitrile, water and mixture of the solvent as above. In a more preferred embodiment the solvent is methanol, tetrahydrofuran or a mixture thereof.

The ratio of the base to the starting material may be increased or decreased accordingly as long as the amount of the acid is an excess to that of the starting material. The preferred ratio thereof is between about 4:1 to about 15:1, and more preferable ratio is between about 6:1 to about 12:1.

The ratio of the compound of Formula Ito the compound of Formula IV may vary depending on the reaction conditions, and may be increased or decreased accordingly. The preferred ratio thereof is between about 1:1 to about 2:1, and more preferable ratio is between about 1:1 to about 1.5:1.

The reaction temperature generally varies depending on the starting material, the solvent and the reagent used in the reaction, and can be changed accordingly. The reaction temperature is preferably, for example, from 0° C. to 70° C., and more preferably, for example, from 10° C. to 40° C.

In one embodiment the reaction conditions comprise imidazole in methanol. In a preferred embodiment, imidazole or sodium acetate can be used as a base in methanol, tetrahydrofuran or a mixture thereof. In a more preferred embodiment, the reaction can be carried out by optionally adding triethylamine to the base and the solvent as stated above.

If the compound of Formula I consists of a mixture of R and S stereoisomers at indicated carbon 1, a mixture of compound 11 and compound 12 will be obtained, as shown in Scheme 9.

The reaction time generally varies depending on the starting material, the solvent and the reagent used in the reaction as well as the reaction temperature and the progress of the reaction, and can be increased or decreased accordingly. The preferred reaction time is, for example, 4 to 120 hours, and more preferably, for example, from 24 to 72 hours.

In Scheme 9, compounds I and IV may be in the form of a salt thereof.

Purification of Compound 12 from a Mixture of Compound 12 and Compound 11

Compound 12 may be obtained in substantial stereochemical purity from a mixture of compound 11 and compound 12 by dissolving the mixture in a suitable solvent or solvent mixture, forming diastereomeric salts by the addition of a chiral carboxylic acid compound, and crystallizing one of the diastereomeric salts from the solution, as shown in Scheme 10, The initially obtained diastereomeric salt can be obtained in greater stereochemical purity by a second recrystallization from a solvent or solvent mixture.

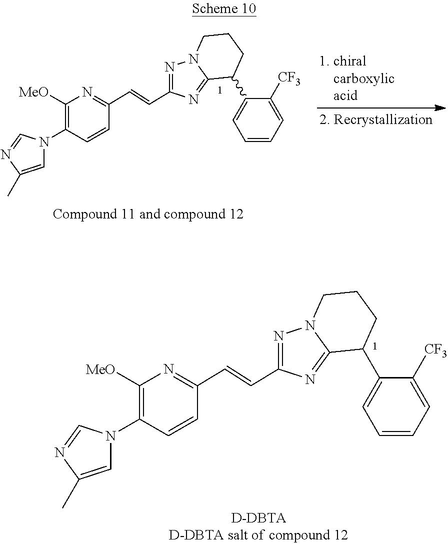

There is no particular restriction on the chiral acid used in the reaction as long as it forms a mixture of diasteromeric salts of compound 11 and 12, but preferred examples of the acid include an acid such as 2,3-bis(benzoyloxy)tartaric acid (DBTA), dipivaloyl tartaric acid (DPTA) and N-(1-Phenylethyl)phthalamic acid (PEPA). In a more preferred embodiment the acid is (2S,3S)-2,3-bis(benzoyloxy)tartaric acid (D-DBTA), (2S,3S)-2,3-bis[(2,2-dimethylpropanoyl)oxy] succinic acid (D-DPTA) and (R)-(+)-N-(1-Phenylethyl)phthalamic acid ((+)-PEPA).

There is no particular restriction on the solvent used in the reaction as long as it dissolves the starting material and each of the diastereomeric salts to some extent, which may be either an organic solvent or a water-containing solvent, but preferred examples of the solvent include a solvent such as toluene, methanol, ethanol, 1-propanol, isopropanol, ethyl acetate, tetrahydrofuran, 1,4-dioxane, N,N-dimethylformamide (DMF), acetonitrile, water and mixture of the solvent as above. In one preferred embodiment the solvent is a mixture of isopropanol and acetonitrile. In another preferred embodiment, the solvent is a mixture of methanol and acetonitrile.

The ratio of the acid to the starting material may be increased or decreased but the preferred ratio is between about 0.5:1 to about 1.3:1. The preferred ratio thereof is between about 0.5:1 to about 0.6:1.

The reaction temperature generally varies depending on the starting material, the solvent and the reagent used in the reaction, and can be changed accordingly. The reaction temperature is preferably, for example, from 0° C. to 70° C., and more preferably, for example, from 0° C. to 50° C.

In the procedure of the step, the second recrystallization can be used in order to improve enantiomeric purity.

A preferred condition for the initial crystallization is the use of a co-solvent mixture of 2-propanol and acetonitrile, and use of (2S,3S)-2,3-bis(benzoyloxy)tartaric acid as the chiral carboxylate. Another preferred condition for the initial crystallization is use of a co-solvent mixture of methanol and acetonitrile and use of (2S,3S)-2,3-bis(benzoyloxy)tartaric acid as the chiral carboxylate. A preferred condition for the second recrystallization is the use of a 1:1 co-solvent mixture of 2-propanol and acetonitrile. Another preferred condition for the second recrystallization is the use of a 2:1 co-solvent mixture of 2-propanol and acetonitrile.

### MODE FOR CARRYING THE INVENTION

The following abbreviations are used in the following examples.

D-DBTA: D-Dibenzoyltartaric acid
Other Names: (2S,3S)-2,3-bis(benzoyloxy)succinic acid

D-DPTA: D-Dipivaloyltartaric acid
Other Names: (2S,3S)-2,3-bis[(2,2-dimethylpropanoyl)oxy]succinic acid

(+)-PEPA: (+)-N-(1-Phenylethyl)phthalamic acid
Other Names: 2-{[(1R)-1-phenylethyl]carbamoyl}benzoic acid

AcCl: Acetyl chloride

DMF: N,N-Dimethylformamide

THF: Tetrahydrofuran

EDC: 1-Ethyl-3-(3-dimethylaminopropyl)carbodiimide hydrochloride

HOBT: 1-Hydroxybenzotriazole

IPEA: Diisopropylethylamine

IPA: 2-Propanol

tert-: Tertiary

Chromatography was performed using BW-300 manufactured by Fuji Silysia Chemical Ltd. as a carrier unless otherwise specified.

LC-MS: High performance liquid chromatography for preparative isolation of a target compound using mass spectroscopy. As an elution solvent, a 10% to 99% linear gradient system of water containing 0.1% trifluoroacetic acid and acetonitrile containing 0.1% trifluoroacetic acid was used.

The sign of optical rotation for each of the purified enantiomers compound 11 and compound 12 was measured in a polarimeter using standard methods known to those in the art.

Diastereomeric excess (de) measurements were measured by a chiral HPLC method:

Column: Chiral Tech IB (150×4.6 mm)

Mobile Phase EtOH/Hexane=40/60

Flow rate: 1 ml/min, isocratic for 15 min

Temperature: 25 degree C.

UV=254 nm

### Example 1

### Synthesis of (+)-2-{(E)-2-[6-methoxy-5-(4-methyl-1H-imidazol-1-yl)pyridin-2-yl]vinyl}-8-(2-trifluoromethylphenyl)-5,6,7,8-tetrahydro-[1,2,4]triazolo[1,5-a]pyridine (Compound 11) and (−)-2-{(E)-2-[6-methoxy-5-(4-methyl-1H-imidazol-1-yl)pyridin-2-yl]vinyl}-8-(2-trifluoromethylphenyl)-5,6,7,8-tetrahydro-[1,2,4]triazolo[1,5-a]pyridine (Compound 12) by the process of Scheme 2 and separation by chiral chromatography of the enantiomeric mixture

### (1). Synthesis of 1-amino-3-(2-trifluoromethylphenyl)piperidin-2-one (1)

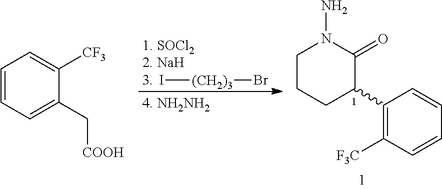

Thionyl chloride (2.72 mL) was added to a solution of 2-trifluoromethylphenylacetic acid (1.9 g) in methanol (38 mL), and the reaction solution was stirred at room temperature for three hours. The reaction solution was concentrated under reduced pressure. The resulting residue was diluted with DMF. Sodium hydride (containing 40% of mineral oil, 410 mg) was added under ice-cooling, and the reaction solution was stirred for 10 minutes. The reaction solution was further stirred at room temperature for 30 minutes and then ice-cooled again. 1-Chloro-3-iodopropane (1.02 mL) was added to the reaction mixture, and the reaction solution was stirred at room temperature overnight. Water and ethyl acetate were added to the reaction mixture and the organic layer was separated. The resulting organic layer was washed with saturated aqueous sodium chloride, dried over anhydrous magnesium sulfate and then concentrated under reduced pressure. The resulting residue was diluted with ethanol (26.6 mL). Hydrazine monohydrate (7.6 mL) was added, and the reaction solution was stirred at room temperature for two hours and then at 60° C. for further three hours. The reaction mixture was concentrated under reduced pressure. Saturated aqueous sodium bicarbonate and ethyl acetate and were added to the residue, and the organic layer was separated. The resulting organic layer was washed with saturated aqueous sodium chloride, dried over anhydrous magnesium sulfate and then concentrated under reduced pressure. The residue was purified by silica gel column chromatography (carrier: Chromatorex NH; elution solvent: heptane-ethyl acetate system) to obtain 1.68 g of the title compound. The property values of the compound are as follows.

ESI-MS; m/z 259 [M⁺+H]. ¹H-NMR (400 MHz; CDCl₃) δ (ppm): 1.82-2.10 (m, 3H), 2.18-2.26 (m, 1H), 3.58-3.76 (m, 2H), 4.07 (dd, J=10.0, 5.6 Hz, 1H), 4.60 (s, 2H), 7.24 (d, J=7.6 Hz, 1H), 7.35 (t, J=7.6 Hz, 1H), 7.51 (t, J=7.6 Hz, 1H), 7.66 (d, J=7.6 Hz, 1H).

### (2). Synthesis of (E)-3-[6-methoxy-5-(4-methyl-1H-imidazol-1-yl)pyridin-2-yl]-N-[2-oxo-3-(2-trifluoromethylphenyl)piperidin-1-yl]acrylamide (3)

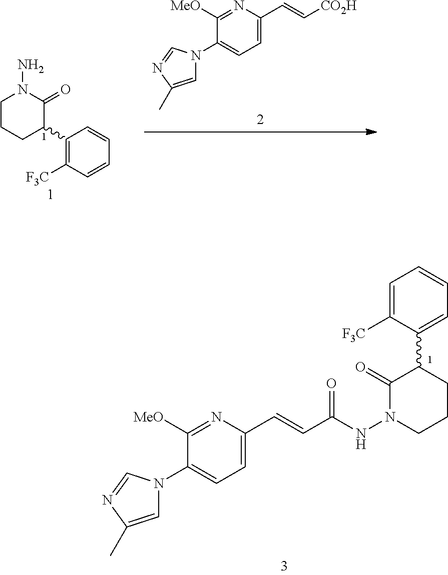

EDC (834 mg), HOBT (588 mg) and IPEA (2.03 mL) were added to a suspension of (E)-3-[6-methoxy-5-(4-methyl-1H-imidazol-1-yl)pyridin-2-yl]acrylic acid trifluoroacetate (2) (1.42 g) and 1-amino-3-(2-trifluoromethylphenyl)piperidin-2-one (1) (750 mg) in DMF (30 mL). The reaction mixture was stirred at room temperature for 14 hours. Then, saturated aqueous sodium bicarbonate and ethyl acetate were added to the reaction solution, and the organic layer was separated. The resulting organic layer was dried over anhydrous magnesium sulfate and then concentrated under reduced pressure. The residue was purified by silica gel column chromatography (carrier: Chromatorex NH; elution solvent: ethyl acetate-methanol system) to obtain 1.23 g of the title compound. The property value of the compound is as follows.

ESI-MS; m/z 500 [M⁺+H].

### (3). Synthesis of (+)-2-{(E)-2-[6-methoxy-5-(4-methyl-1H-imidazol-1-yl)pyridin-2-yl]vinyl}-8(2-trifluoromethylphenyl)-5,6,7,8-tetrahydro-[1,2,4]triazolo[1,5-a]pyridine and (−)-2-{(E)-2-[6-methoxy-5-(4-methyl-1H-imidazol-1-yl)pyridin-2-yl]vinyl}-8-(2-trifluoromethylphenyl)-5,6,7,8-tetrahydro-[1,2,4]triazolo[1,5-a]pyridine

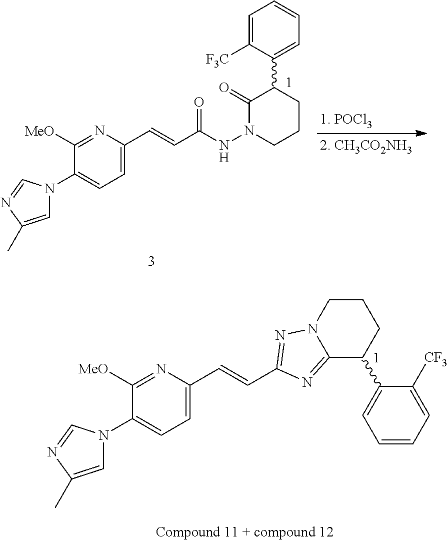

Phosphorus oxychloride (24.2 mL) was added to (E)-3-[6-methoxy-5-(4-methyl-1H-imidazol-1-yl)pyridin-2-yl]-N-[2-oxo-3-(2-trifluoromethylphenyl)piperidin-1-yl]acrylamide (3) (1.2 g). The reaction solution was stirred at 100° C. for one hour and then concentrated under reduced pressure. Subsequently, the residue was diluted with acetic acid (24.2 mL). Then, ammonium acetate (1.9 g) was added and the reaction solution was stirred at 150° C. for two hours. The reaction solution was left to cool to room temperature and then concentrated under reduced pressure. Saturated aqueous sodium bicarbonate and ethyl acetate were added to the resulting residue, and the organic layer was separated. The resulting organic layer was dried over anhydrous magnesium sulfate and then concentrated under reduced pressure. The residue was purified by silica gel column chromatography (carrier: Chromatorex NH; elution solvent: heptane-ethyl acetate system) to obtain a racemate of the title compound (750 mg). The resulting racemate (410 mg) was separated by CHIRALPAK™ IA manufactured by Daicel Chemical Industries, Ltd. (2 cm×25 cm, mobile phase; hexane:ethanol=8:2, flow rate: 10 mL/min) to obtain one of the title enantiomers with a retention time of 28 minutes and positive optical rotation (compound 11; 174 mg), and the other title enantiomer with a retention time of 33 minutes and negative optical rotation (compound 12; 170 mg).

The property values of the title enantiomer with a retention time of 28 minutes (compound 11) are as follows.

¹H-NMR (400 MHz; CDCl₃) δ (ppm): 1.90-2.01 (m, 1H), 2.10-2.35 (m, 2H), 2.29 (d, J=1.2 Hz, 3H), 2.42-2.51 (m, 1H), 4.03 (s, 3H), 4.28-4.41 (m, 2H), 4.70 (dd, J=8.4, 6.0 Hz, 1H), 6.92 (d, J=8.0 Hz, 1H), 6.95 (t, J=1.2 Hz, 1H), 7.01 (d, J=7.6 Hz, 1H), 7.39 (t, J=7.6 Hz, 1H), 7.44 (d, J=16.0 Hz, 1H), 7.45 (d, J=8.0 Hz, 1H), 7.49 (t, J=7.6 Hz, 1H), 7.63 (d, J=16.0 Hz, 1H), 7.72 (d, J=7.6 Hz, 1H), 7.76 (d, J=1.2 Hz, 1H).

The property values of the title enantiomer with a retention time of 33 minutes (compound 12) are as follows.

¹H-NMR (400 MHz; CDCl₃) δ (ppm): 1.90-2.01 (m, 1H), 2.10-2.35 (m, 2H), 2.29 (d, J=1.2 Hz, 3H), 2.42-2.51 (m, 1H), 4.03 (s, 3H), 4.28-4.41 (m, 2H), 4.70 (dd, J=8.4, 6.0 Hz, 1H), 6.92 (d, J+8.0 Hz, 1H), 6.95 (t, J=1.2 Hz, 1H), 7.01 (d, J=7.6 Hz, 1H), 7.39 (t, J=7.6 Hz, 1H), 7.44 (d, J=16.0 Hz, 1H), 7.45 (d, J=8.0 Hz, 1H), 7.49 (t, J=7.6 Hz, 1H), 7.63 (d, J=16.0 Hz, 1H), 7.72 (d, J=7.6 Hz, 1H), 7.76 (d, J=1.2 Hz, 1H).

### Example 2

### Synthesis of 5-Chloro-2-phenylpentanenitrile (9)

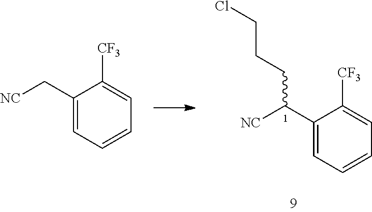

2-(Trifluoromethyl)phenylacetonitrile (12.47 g, 67.3 mmol) was dissolved in THF (87.3 mL) at room temperature under nitrogen atmosphere. The reaction solution was cooled to −10° C. Then, potassium tert-butoxide (7.93 g, 70.7 mmol) was added to the reaction solution and the reaction mixture was stirred at −10° C. for 10 minutes. 1-Bromo-3-chloropropane (6.99 mL, 70.7 mmol) was added dropwise to the reaction mixture over 14 minutes, and the reaction mixture was stirred at 0° C. for 2 hours. The reaction was quenched with 10% NH₄Cl aq. (8.6 mL). After the mixture was stirred, the aqueous layer was separated. The organic layer was concentrated under the reduced pressure to obtain the title compound (23.24 g). The yield was calculated as over 99% by HPLC external standard method.

¹H-NMR (400 MHz, CDCl₃) δ (ppm): 2.18-1.88 (m, 4H), 3.58 (m, 2H), 4.18 (m, 1H), 7.47 (t, 1H, J=7.6 Hz), 7.65 (t, 1H, J=7.6 Hz), 7.71 (m, 2H).

### Example 3

### Synthesis of Ethyl 5-chloro-2-phenylpentanimidate hydrochloride (10)

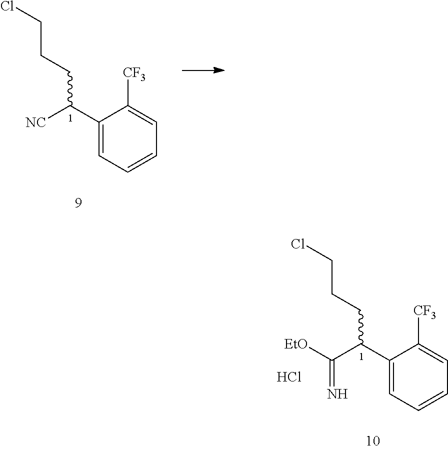

5-Chloro-2-phenylpentanenitrile (9) (2.0 g, 7.64 mmol) was dissolved in ethanol (5.36 mL, 91.72 mmol) at room temperature under nitrogen atmosphere. Then, the solution was cooled to 0° C. Acetyl chloride (4.34 mL, 61.14 mmol) was added dropwise to the solution, and the reaction mixture was stirred at room temperature for 67 hours. The reaction mixture was cooled to 10° C. Traces of seed crystal of the title compound and tert-butylmethylether (hereinafter referred to as “MTBE”) (40 mL) were added to the reaction mixture and the reaction mixture was stirred. The solid was collected by filtration, washed with MTBE to obtain the title compound (2.14 g, 81.6% yield).

¹H-NMR (400 MHz, CDCl₃) δ (ppm): 1.38 (t, 3H, J=7.2 Hz), 1.78-1.65 (m, 1H), 1.95-1.83 (m, 1H), 2.43-2.32 (m, 1H), 2.65-2.50 (m, 1H), 3.62-3.55 (m, 2H), 4.47 (t, 1H, J=8 Hz), 4.65 (q, 2H, J=7.2 Hz), 7.47 (t, 1H, J=8.0 Hz), 7.66 (t, 1H, J=8.0 Hz), 7.71 (d, 1H, J=8.0 Hz), 7.85 (d, 1H, J=8.0 Hz), 12.05 (br s, 1H), 12.58 (br s, 1H).

### Example 4

### Synthesis of 6-bromo-2-methoxy-3-(4-methyl-1H-imidazol-1-yl)pyridine (compound 4)

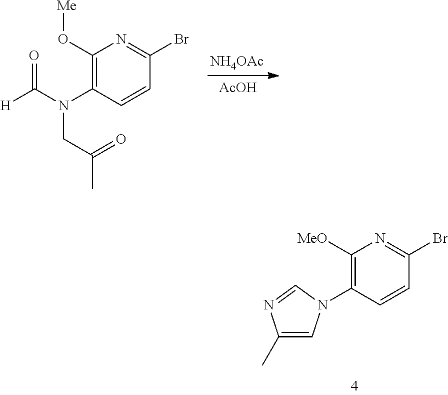

A suspension of ammonium acetate (267 g) and N-(6-bromo-2-methoxypyridin-3-yl)-N-(2-oxopropyl)formamide (199 g) in glacial acetic acid (400 ml) was stirred at 130° C. for one hour and 10 minutes. The reaction solution was returned to room temperature. Ethyl acetate and ice water were added to the reaction solution, and the reaction solution was ice-cooled. Then, concentrated aqueous ammonia (500 ml) was added dropwise and then the organic layer was separated. The resulting organic layer was sequentially washed with water and brine and dried over anhydrous magnesium sulfate. Then, the organic layer was purified by short silica gel column chromatography (carrier: Wakogel C-200; elution solvent: ethyl acetate). The eluted fraction was concentrated. The resulting residue was triturated with ethyl acetate and tert-butyl methyl ether and dried under reduced pressure to obtain 107.7 g of the title compound.

Then, the trituration mother liquor was concentrated. The resulting residue was purified by silica gel column chromatography (carrier: Wakogel C-200; elution solvent toluene-ethyl acetate system). The target fraction was concentrated. The resulting residue was triturated with tert-butyl methyl ether and dried under reduced pressure to obtain 12.9 g of the title compound.

The property values of the compound are as follows.

¹H-NMR (400 MHz; CDCl₃) δ (ppm); 2.29 (d, J=0.8 Hz, 3H), 4.03 (s, 3H), 6.92 (dd, J=1.2, 0.8 Hz, 1H), 7.16 (d, J=8.0 Hz, 1H), 7.40 (d, J=8.0 Hz, 1H), 7.73 (d, J=1.2 Hz, 1H). ESI-MS; m/z 268 [M⁺+H].

### Example 5

### Synthesis of tert-Butyl 2-{(2E)-3-[6-methoxy-5-(4-methyl-1H-imidazol-1-yl)pyridin-2-yl]prop-2-enoyl}hydrazinecarboxylate (compound 6)

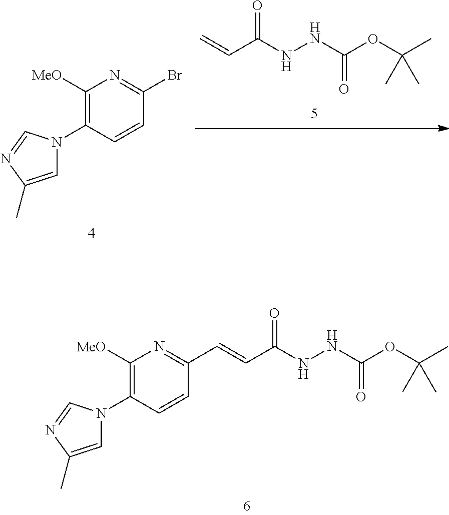

DMF (52 mL) was added to 6-Bromo-2-methoxy-3-(4-methyl-1H-imidazol-1-yl)pyridine (13.0 g, 48.5 mmol) and the tert-Butyl 2-acryloylhydrazinecarboxylate (9.9 g, 53.3 mmol) at room temperature under nitrogen atmosphere, And the mixture was stirred at 50° C. for 10 minutes. Tri(o-tolyl)phosphine (885 mg, 2.90 mmol), Palladium (II) acetate (327 mg, 1.45 mmol) and N,N-diisopropylethylamine (12.7 mL, 72.7 mmol) were added to the mixture, and the reaction mixture was stirred at 100° C. for 4 hours. The reaction mixture was cooled to room temperature and filtrated through Celite. The residue was washed twice with DMF (6 mL). Water (104 mL) was added dropwise to the filtrate at room temperature over 10 minutes. The mixture was stirred at room temperature for 15 hours. After the mixture was filtrated, the residue was washed with water/DMF=2:1(30 mL) and MTBE (30 mL). The obtained solid was suspended in MTBE (50 mL) at room temperature for 2 hours, filtrated and dried under the reduced pressure to obtain the title compound (15.8 g, 87% yield), ¹H-NMR (400 MHz, CDCl₃) δ (ppm): 1.50 (s, 9H), 2.28 (d, J=1.2 Hz, 3H), 4.03 (s, 3H), 6.83 (brs, 1H), 6.97-7.02 (m, 3H), 7.51 (d, Hz, 1H), 7.59 (d, J=15.2 Hz, 1H), 7.82 (s, 1H), 8.01 (br s, 1H).

### Example 6

### Synthesis of (2E)-3-[6-Methoxy-5-(4-methyl-1H-imidazol-1-yl)pyridin-2-yl]acrylohydrazide dihydrochloride (compound 7)

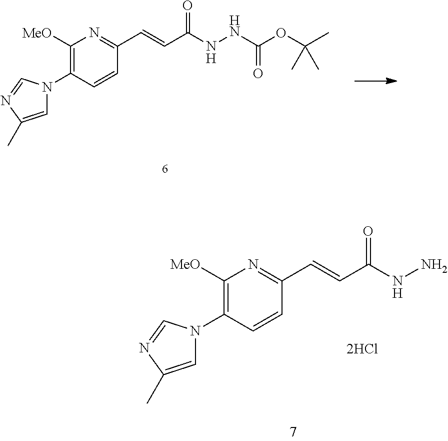

Conc. HCl (5.85 mL) was added to the suspension of tert-Butyl 2-{(2E)-3-[6-methoxy-5-(4-methyl-1H-imidazol-1-yl)pyridin-2-yl]prop-2-enoyl}hydrazinecarboxylate (1.17 g, 3.13 mmol) in methanol (5.85 mL) with an ice-bath cooling. The reaction mixture was stirred at room temperature for 30 minutes. 1-Butanol (5.85 mL) and MTBE (5.85 mL) were added to the reaction mixture, and the mixture was stirred for 20 minutes with an ice-bath cooling. The mixture was filtrated, and the residue was washed with 1-butanol-MTBE (2:8) (5.85 mL) and dried under the reduced pressure to obtain the title compound (937 mg, 78.2% yield).

¹H NMR (400 MHz, d₆-DMSO) δ (ppm): 2.36 (d, J=0.8 Hz, 3H), 3.82 (brs, 2H), 4.04 (s, 3H), 7.28 (d, J=15.2 Hz, 1H), 7.54 (d, J=8.0 Hz, 1H), 7.70 (d, J=15.2 Hz, 1H), 7.83 (d, J=1.6 Hz, 1H), 8.15 (d, J=7.6 Hz), 9.44 (d, 1H), 11.56 (s, 1H).

### Another synthetic route for (2E)-3-[6-Methoxy-5-(4-methyl-1H-imidazol-1-yl)pyridin-2-yl]acrylohydrazide dihydrochloride (compound 7)

2-{(2E)-3-[6-methoxy-5-(4-methyl-1H-imidazol-1-yl)pyridin-2-yl]prop-2-enoyl}hydrazinecarboxylate (58.62 g) was added to the mixture of 1-propanol (415 mL) and conc. HCl (180 mL) at 45° C. The reaction mixture was stirred at 45° C. for 25 minutes. 1-Propanol (300 mL) was added, and stirred with an ice-bath cooling. The mixture was filtrated, and the residue was washed with 1-propanol (150 mL) and dried under the reduced pressure to obtain the title compound (47.26 g, 87% yield).

¹H NMR spectrum was identical as above.

### Example 7

### Synthesis of 2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine (compound 11/compound 12)

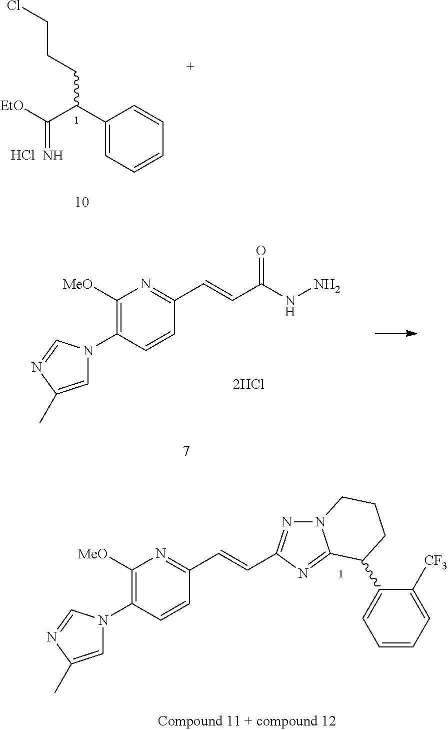

Imidazole (4.75 g, 69.7 mmol) and ethyl 5-chloro-2-phenylpentanimidoate hydrochloride (2.00 g, 5.81 mmol) were added the solution of (2E)-3-[6-Methoxy-5-(4-methyl-1H-imidazol-1-yl)pyridin-2-yl]acrylohydrazide dihydrochloride in methanol (10 mL) at 0° C. under nitrogen atmosphere. The reaction mixture was stirred at 30° C. for 40 hours. The reaction mixture was adjusted to the pH6.5 with 5N HCl aq., and extracted with ethyl acetate (22 mL). The organic layer was washed with water (4 mL), concentrated under the reduced pressure and azeotroped with 2-propanol under the reduced pressure to obtain the title compound (2.4 g, 86% yield). Traces of seed crystal of the title compound which was obtained by the method of Scheme 2 was added to the solution of the crude title compound in 2-propanol (10 mL), and the mixture was stirred at room temperature for 13.5 hours. The suspension was stirred for 2 hours with an ice-bath cooling. The solids were collected by filtration and washed with 2-propanol and dried under the reduced pressure to obtain the title compound as a mixture of enantiomers (1.55 g, 56% yield). ¹H NMR (400 MHz; CDCl₃) δ (ppm): 1.91-2.01 (1H, m), 2.10-2.21 (1H, m), 2.23-2.28 (1H, m), 2.29 (3H, d, J=1.0), 2.43-2.50 (1H, m), 4.03 (3H, s), 429 4.40 (2H, m), 4.71 (1H, dd, J=6.0, 8.4 Hz), 6.93 (1H, d, J=7.8 Hz), 6.95 (1H, dd, J=1.0 Hz), 7.02 (1H, d, J=7.8 Hz), 7.39 (1H, dd, J=7.6 Hz), 7.43 (1H, d, J=15.6 Hz), 7.46 (1H, d, J=7.8 Hz), 7.49 (1H, dd, J=7.3 Hz), 7.64 (1H, d, J=15.6 Hz), 7.73 (1H, d, J=7.1 Hz), 7.76 (1H, d, J=1.2 Hz).

### Example 8

### Synthesis of (−)-(8S)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine-(2S,3S-2,3-bis(benzoyloxy)tartaric acid (1/1)(D-DBTA salt of compound 12)

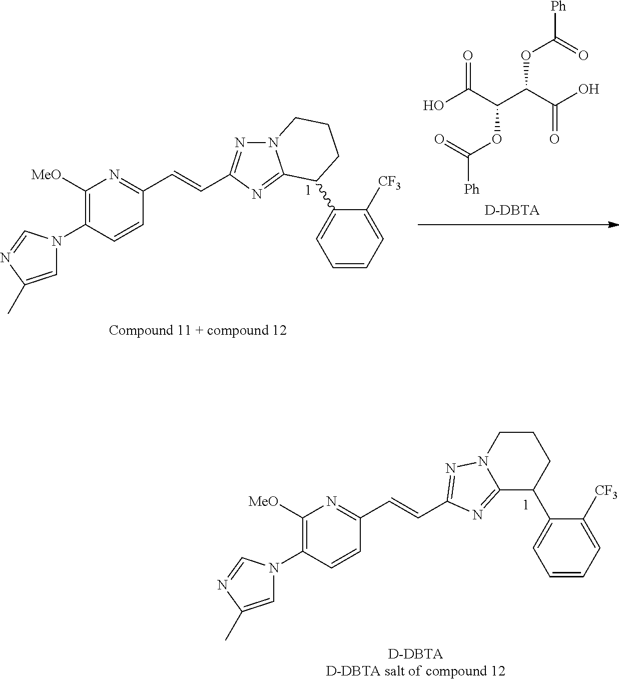

2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine (100 mg, 0.208 mmol) was dissolved in the mixture of 2-propanol (1.6 mL) and acetonitrile (2.0 mL) at 45° C., and the solution of D-DBTA (89.5 mg, 0.250 mmol) in acetonitrile (1.6 mL) was added. Traces of seed crystal of the title compound which was obtained by the same method except the temperature of the solvent was 60° C. and without seed crystal was added to the solution at 33° C., and the mixture was stirred at room temperature for 18 hours. The solids were collected by filtration, washed with acectonitrile/2-propanol=2/1 (0.5 mL) and dried at 50° C. under the reduced pressure to obtain the title compound (62.3 mg, 35.7% yield, 90.7% de). The title compound (50.7 mg, 90.7% de) was suspended in acectonitrile/2-propanol=1/1 (0.5 mL), and the mixture was stirred at 80° C. for 25 minutes, and then stirred at room temperature for 15 hours. The solids were collected by filtration and dried at 50° C. under the reduced pressure to obtain the title compound (35.9 mg, 70.8% yield, 98.1% de)

¹H NMR (400 MHz, d₆-DMSO) δ (ppm): 1.90-2.00 (1H, m), 2.12-2.20 (1H, m), 2.15 (3H, s), 2.27-2.32 (2H, m), 3.98 (3H, s), 4.27-4.31 (2H, m), 4.48-4.52 (1H, dd, J=5.9, 9.5 Hz), 5.84 (2H, s), 7.24-7.34 (4H, m), 7.44-7.51 (2H, m), 7.56-7.63 (5H, m), 7.69-7.80 (4H, m), 7.96-8.00 (5H, m).

### Example 9

### Synthesis of (−)-(8S)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]-triazolo[1,5-a]pyridine (compound 12)

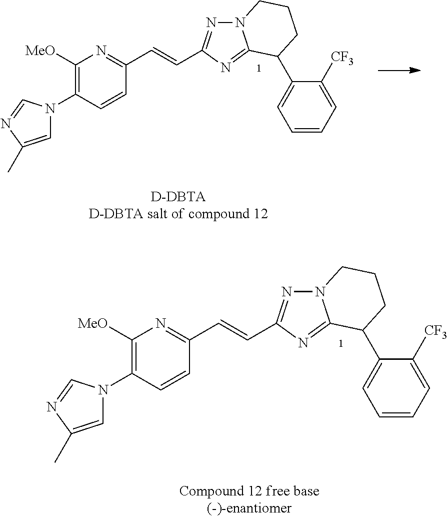

(−)-(8S)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine-(2S,3S)-2,3-bis(benzoyloxy)tartaric acid (1/1) (20 mg, 0.024 mmol) was added to the mixed solution of ethyl acetate (0.1 mL) and 5N HCl aq. (0.1 mL), and the organic layer was separated. Ethyl acetate (0.2 mL) and 5N sodium hydroxide aq. (0.1 mL) were added to the aqueous layer, and the organic layer was separated. The organic layer was washed twice with water (0.1 mL), and dried under the reduced pressure to obtain the title compound (11.5 mg, 99.9% yield), negative optical rotation.

### Example 10

### Synthesis of (−)-(8S)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine-(2S,3S)-2,3-bis[(2,2-dimethylpropanoyl)oxy]succinic acid (1/1) (D-DPTA salt of compound 12)

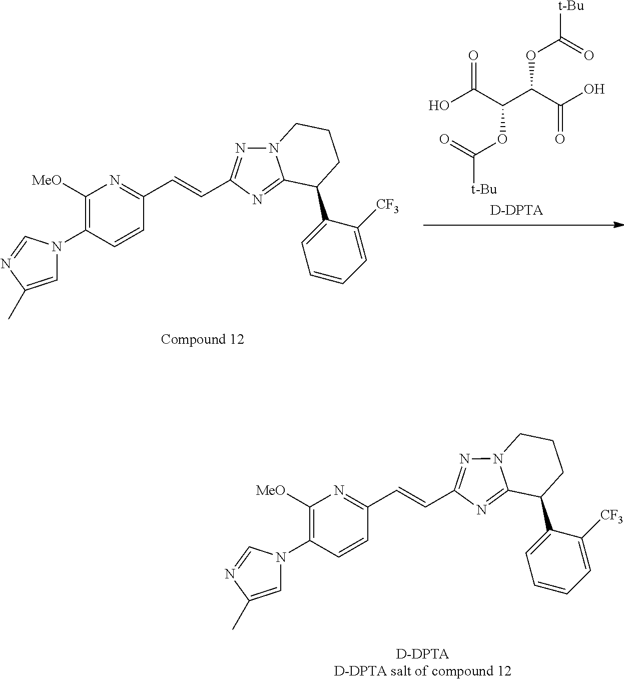

(−)-(8S)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine (48.0 mg, 0.10 mmol) and D-DPTA (31.8 mg, 0.10 mmol) were stirred in 2-propanol (1.0 mL) for 2.5 hours. The solids were collected by filtration, washed with 2-propanol and heptane, and dried at 50° C. under the reduced pressure to obtain the title compound (74.6 mg, 93.4% yield).

¹H NMR (400 MHz, d₆-DMSO) δ (ppm): 1.15 (18H, s), 1.90-2.00 (1H, m), 2.12-2.20 (2H, m), 2.15 (3H, s), 2.27-2.32 (1H, m), 3.98 (3H, s), 4.25-4.34 (2H, m), 4.49-4.53 (1H, dd, J=6.1, 9.3 Hz), 5.41 (2H, s), 7.23-7.33 (4H, m), 7.44-7.51 (2H, m), 7.61 (1H, t, J=7.3 Hz), 7.75-7.79 (2H, m), 7.93 (1H, d, J=1.2 Hz).

### Example 11

### Synthesis of (−)-(8S)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine-(2S,3S)-2,3-bis[(2,2-dimethylpropanoyl)oxy]succinic acid (1/1) (D-DPTA salt of compound 12 (from mixture of compound 11 and compound 12))

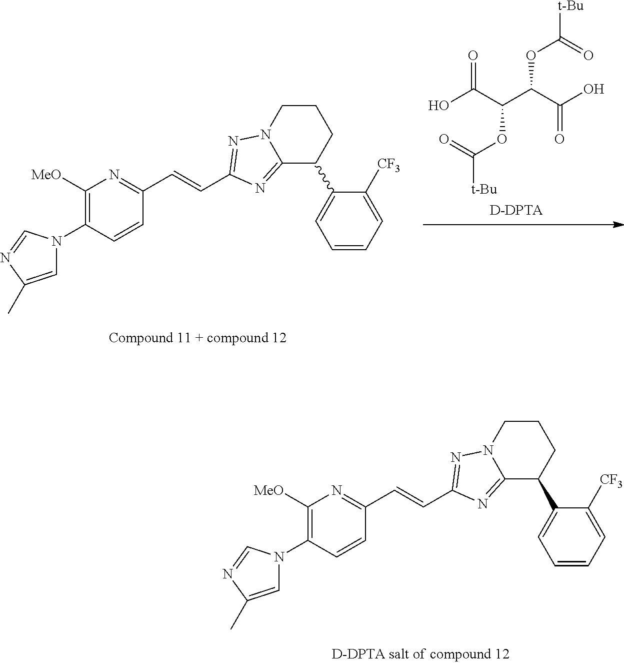

2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine (192.0 mg, 0.40 mmol) was dissolved in the mixture of 2-propanol (0.64 mL) and acetonitrile (0.64 mL) at 50° C., and the solution of D-DPTA (76.4 mg, 0.24 mmol) in acetonitrile (0.64 mL) was added. Trace of seed crystal of the title compound obtained from example 10 was added to the solution, and the mixture was cooled to 10° C. The solids were collected by filtration, washed with the mixture of acectonitrile/2-propanol=3/1 (1.5 mL), and dried at 50° C. under the reduced pressure to obtain the title compound (139.6 mg, 43.7% yield, 86.3% de).

### Example 12

### Synthesis of (−)-(8S)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine (compound 12 (from D-DPTA salt of compound 12))

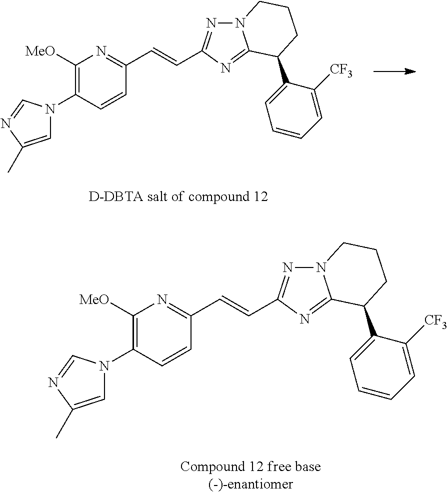

(−)-(8S)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine-(2S,3S)-2,3-bis[(2,2-dimethylpropanoyl)oxy]succinic acid (1/1) (20 mg, 0.0250 mmol) was added to the mixed solution of ethyl acetate (0.2 mL) and 5N HCl aq. (0.1 mL), and the organic layer was separated. Isopropyl acetate (0.18 mL), methanol (0.02 mL) and 5N sodium hydroxide aq. (0.11 mL) were added to the aqueous layer, and the organic layer was separated. The organic layer was washed thrice with water (0.2 mL×2, 0.1 mL×1), and dried under the reduced pressure to obtain the title compound (11.0 mg, 91.4% yield), negative optical rotation.

### Example 13

### Synthesis of (−)-(8S)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine-2-{[(1R)-1-phenylethyl]carbamoyl}benzoic acid (1/1) ((+)-PEPA salt of compound 12)

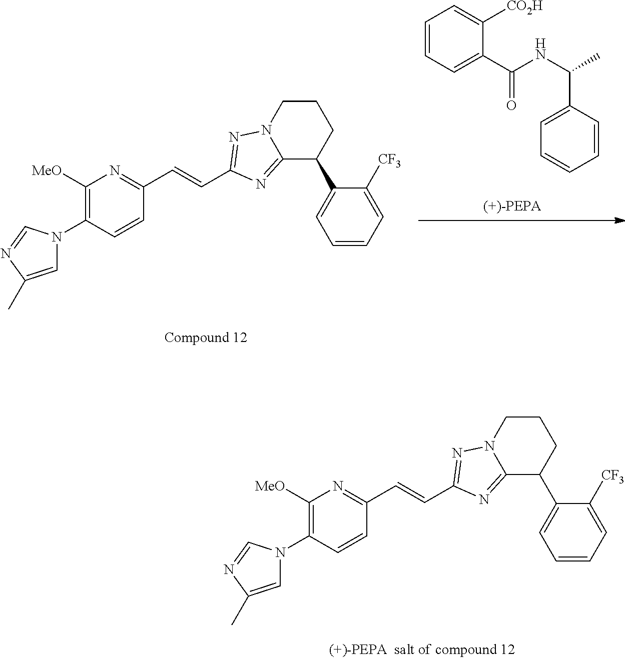

(−)-(8S)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine (48.0 mg, 0.10 mmol) and (+)-PEPA (53.9 mg, 0.20 mmol) were dissolved in 2-propanol (1.5 mL) at 50° C., and the mixture was cooled to room temperature. The solids were collected by filtration, washed with 2-propanol, and dried at 50° C. under the reduced pressure to obtain the title compound (49.5 mg, 66.0% yield).

1H NMR (400 MHz, CDCl₃) δ (ppm): 1.41 (3H, d, J=4.9 Hz), 1.90-2.00 (1H, m), 2.12-2.20 (2H, m), 2.14 (3H, s), 2.25-2.35 (1H, m), 3.98 (3H, s), 4.27-4.31 (2H, m), 4.49-4.53 (1H, dd, J=6.1, 9.3 Hz), 5.06-5.14 (1H, m), 7.19-7.33 (6H, m), 7.39-7.63 (8H, m), 7.75-7.78 (3H, m), 7.87 (1H, d, J=1.5 Hz), 8.69 (1H, d, J=8.8 Hz).

### Example 14

### Synthesis of (−)-(8S)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine-2-{[(1R)-1-phenylethyl]carbamoyl}benzoic acid (1/1) ((+)-PEPA, salt of compound 12 (from mixture of compound 11 and compound 12))

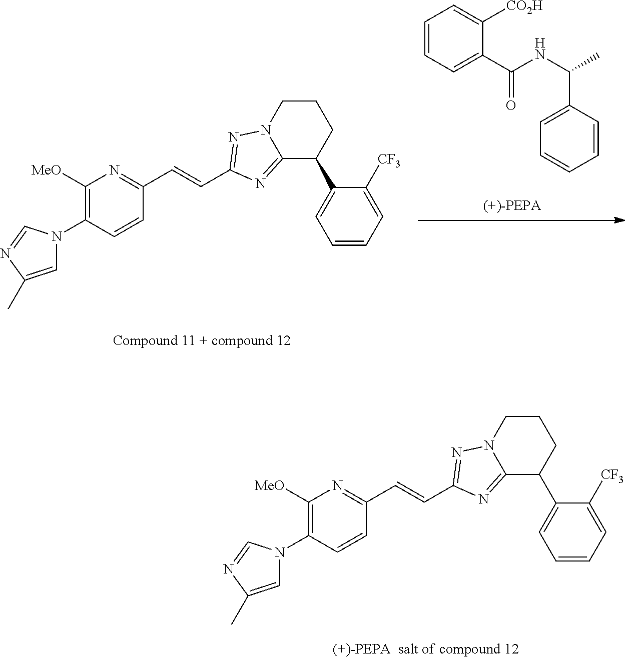

2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine (96.1 mg, 0.20 mmol) and (+)-PEPA (53.9 mg, 0.20 mmol) were dissolved in 2-propanol (11.0 mL) at 40° C., and the mixture was cooled to room temperature. The solids were collected by filtration, washed with 2-propanol, and dried at 50° C. under the reduced pressure to obtain the title compound (47.0 mg, 31.3% yield, 93.2% de).

### Example 15

### Synthesis of (−)-(8S)-2-{(E)-2-[6-Methyoxy-5(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine (compound 12 (from (+)-PEPA salt of compound 12))

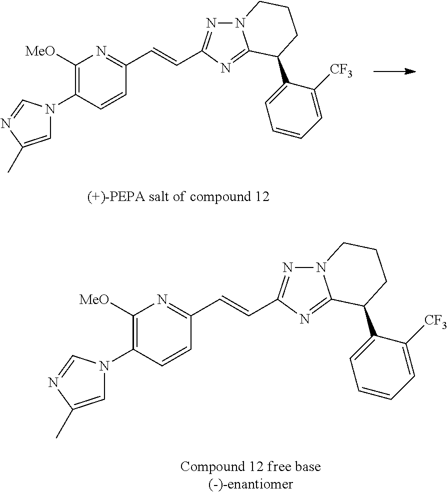

(−)-(8S)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine-2-{[(1R)-1-phenylethyl]carbamoyl}benzoic acid (100 mg, 0.133 mmol) was added to the mixed solution of ethyl acetate (1.0 mL) and 5N HCl aq. (0.5 mL), and the organic layer was separated. Isopropyl acetate (0.9 mL), methanol (0.1 mL) and 5N sodium hydroxide aq. (0.55 mL) were added to the aqueous layer, and the organic layer was separated. The organic layer was washed thrice with water (1.0 mL×2, 0.5 mL×1), and dried under the reduced pressure to obtain the title compound (50.8 mg, 79.3% yield), negative optical rotation.

### Example 16

### Synthesis of 5-Chloro-2-(2-trifluoromethyl-phenyl)-pentanoic acid

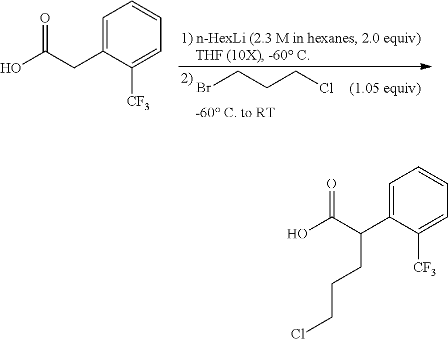

A 1 L, 3-necked round bottom flask was charged with 20.4 g of 2-trifluoromethylphenylacetic acid and 200 mL of anhydrous THF under a nitrogen atmosphere, and the mixture was cooled to −60° C. in a dry ice/IPA bath. n-Hexyllithium (2.3 M in hexane; 43 mL) was added dropwise, maintaining the internal temperature below −50° C. The mixture was stirred at −60° C. for 1 h. Additional n-hexyllithium (44 mL) was added dropwise, again maintaining the internal temperature below −50° C. The resulting yellow solution was stirred for 1 h at −60° C., then 13 mL of 1-bromo-3-chloropropane was added dropwise. After 3 h, the mixture was allowed to stir with warming to room temperature overnight. The mixture was cooled to 0° C. and treated with 300 mL of 1N NaOH solution, maintaining the internal temperature below 15° C. The mixture was stirred for 10 min after addition and then the phases were split. The aqueous phase was cooled to 0° C. and 6N HCl was added to adjust the pH to 2-3, again maintaining the internal temperature below 15° C. The solution was extracted with toluene (200 mL). The toluene phase was washed with water (2×80 mL). The organic phase was dried (Na₂SO₄), filtered, and concentrated by rotary evaporation to afford 26.9 g of product (98%).

¹H NMR (400 MHz, CDCl3): δ 1.65 (m, 1H); 1.82 (m, 1H); 1.93 (m, 1H); 2.32 (m, 1H); 3.49 (m, 2H); 4.09 (m, 1H); 7.41 (m, 1H); 7.59 (m, 2H); 7.70 (m, 1H).

### Example 17

### Synthesis of 5-Chloro-2-(2-trifluoromethyl-phenyl)-pentanoic acid amide

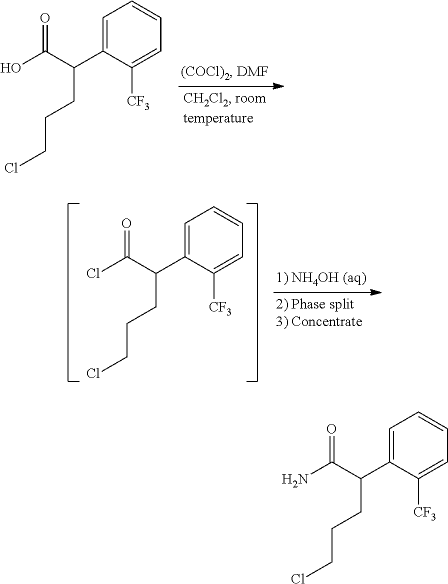

A 100 mL, round bottom flask was charged with a solution of 5.07 g (18.1 mmol) of 5-chloro-2-(2-trifluoromethyl-phenyl)-pentanoic acid in dichloromethane (50 mL). Oxalyl chloride (1.61 mL, 19.0 mmol, 1.05 equivalent) was added. The flask was equipped with a scrubber containing 1N NaOH and DMF (70 uL, 0.05 equiv) was added. The reaction mixture was allowed to stir for 12 h at room temperature. The acid chloride solution was cooled to 0° C. in an ice bath. To the cooled solution was charged dropwise, 22 mL of aqueous NH₄OH solution (28-30 wt % ammonia) with rapid stirring. Addition was conducted at such a rate as to maintain the internal temperature at 15° C. Once the internal temperature returned to 5-7° C., the mixture was warmed to room temperature and stirred for 1 h. Water (25 mL) was added. The mixture was stirred for 20 mins, and the phases were split. The lower organic phase was concentrated to yield the product. ¹H NMR (400 MHz, CDCl3): □ 1.65 (m, 1H); 1.80-2.00 (m, 2H); 2.28 (m, 1H); 3.52 (m, 2H); 3.83 (m, 1H); 5.35-5.58 (br, 2H); 7.38 (m, 1H); 7.57 (m, 1H); 7.65-7.74 (m, 1H).

### Example 18

### Synthesis of 5-Chloro-2-(2-trifluoromethyl-phenyl)-pentanimidic acid ethyl ester

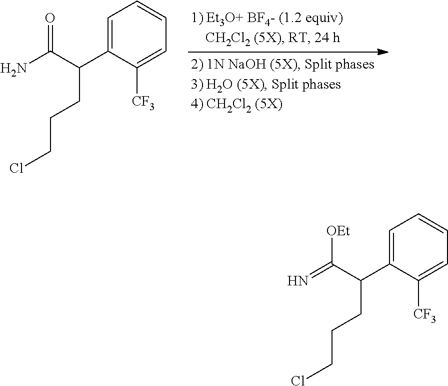

A 25 mg round-bottom flask was charged with triethyloxonium tetrafluoroborate (0.851 g, 4.48 mmol, 1.24 equiv). The solid was dissolved in dichloromethane (1.0 mL). To this solution was charged 7.45 g of a 13.6 wt % solution of 5-chloro-2-(2-trifluoromethyl-phenyl)-pentanoic acid amide in dichloromethane (equivalent to 1.014 g of the amide, 3.62 mmol, 1.0 equiv). The resulting mixture was allowed to stir under nitrogen for 24 h at room temperature. The mixture was treated with 1N NaOH. (5 mL, 5.0 mmol, 1.38 equiv) and the biphasic mixture allowed to stir for 10 mins. The layers were separated and the organic phase was washed 1× with water (5 mL). Dichloromethane (5 mL) was added and the solution concentrated to dryness to provide the product as an oil.

¹H NMR (400 MHz, CDCl3): δ 1.28 (t., 3H); 1.58-1.69 (m, 1H); 1.75-1.87 (m, 1H); 1.90-2.01 (m, 1H); 2.18-2.28 (m, 1H); 3.48-3.56 (m, 2H); 3.92-3.98 (t, 1H); 4.14 (q, 2H); 7.35-7.43 (m, 1H); 7.55-7.62 (m, 2H); 7.69 (d, 1H).

### Example 19

### Synthesis of Ethyl 5-Chloro-2-(2-trifluoromethyl-phenyl)-pentanimidate methylsulfate

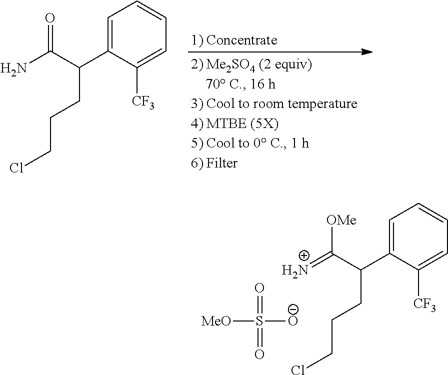

A 25 mL round-bottom flask was charged with 6.6 g of a 13.6 wt % solution of 5-chloro-2-(2-trifluoromethyl-phenyl)-pentanoic acid amide in dichloromethane (equivalent to 0.898 g of amide, 3.2 mmol, 1.0 equiv). The mixture was concentrated to near dryness by rotary evaporation. Dimethyl sulfate (0.64 mL, 6.72 mmol, 2.10 equiv) was added. The flask was equipped with a reflux condenser and nitrogen inlet and immersed in an oil bath. The mixture was heated to 70° C. and aged at this temperature for 16 h. The mixture was cooled to RT and MTBE (5 mL) was added. The solution was cooled to 0° C. and aged at this temperature for 1 h, during which time a white solid precipitate was formed. The mixture was filtered at 0° C. and the wet cake was washed with cold (0° C.) MTBE (2×0.5 mL) and dried. The methylsulfate salt was isolated in 70% yield (0.916 g) as a white solid.

¹H NMR (400 MHz, CDCl3): δ 1.62-1.74 (m, 1H); 1.84-1.96 (m, 1H); 2.31-2.46 (m, 2H); 3.52-3.60 (m, 2H); 3.76 (s, 3H); 4.25 (s, 3H); 4.55-4.58 (m, 1H); 7.46-7.52 (t, 1H); 7.64-7.75 (m, 3H).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the spirit and scope of the present invention. All such modifications are intended to be within the scope of the claims appended hereto.

All patents and publications cited above are hereby incorporated by reference.

### INDUSTRIAL APPLICABILITY

The present invention provides a new synthetic methods for preparing compounds such as compound 12 which is a nonpeptidic compound potently inhibiting production of Aβ42 from APP. Also, the present invention provides an improved method for synthesizing intermediates for the preparation of compounds such as compound 12, and for the preparation of substantially stereochemically pure compounds of the type of compound 12 from stereoisomeric mixtures.

1. A process for preparing compound 12 ((−)-2-{(E)-2-[6-Methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine) in substantial stereochemical purity, comprising the steps of:
a). forming a mixture of compound 11 and compound 12 by reacting a compound of Formula I with a compound of Formula IV as shown below: 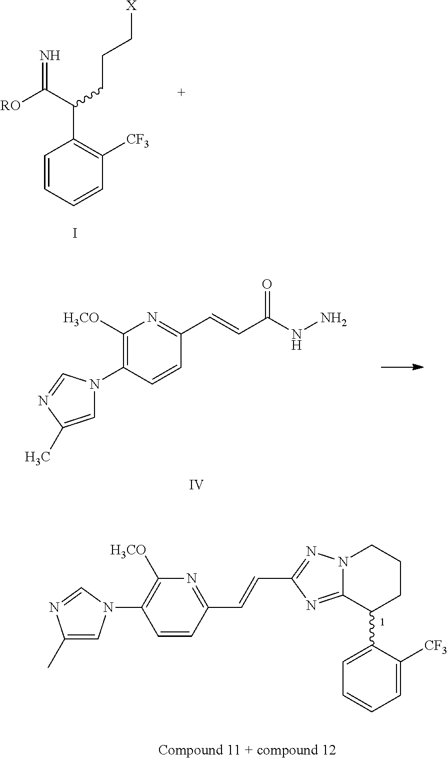
wherein X is a leaving group; R is C1-C6 branched or unbranched alkyl group, or C2-C6 branched or unbranched alkenyl group; and the stereochemistry at carbon 1 is a mixture of R and S isomers
b). forming a mixture of diastereomeric salts of compound 11 and compound 12 by treating the mixture of compound 11 and compound 12 with a chiral carboxylic acid compound;
c). crystallizing the diastereomeric salt formed of compound 12 from a solution of diastereomeric salts formed of compound 11 and compound 12; and
d). forming compound 12 from the obtained diastereomeric salt of compound 12.

2. A process for preparing a mixture of compound 11 and compound 12, comprising the step of reacting a compound of Formula I or a salt thereof with a compound of Formula IV or a salt thereof as shown below: 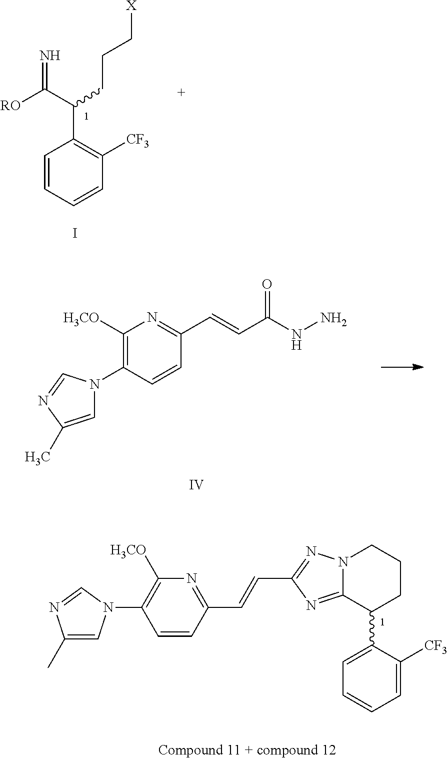
wherein X, R and the stereochemistry at carbon 1 are as defined in claim 1.

3. The process according to claim 1 wherein the reaction is carried out in methanol or tetrahydrofuran or a mixture thereof in the presence of imidazole or sodium acetate, optionally followed by the addition of triethylamine.

4. A process for preparing compound 12 ((−)-2-{(E)-2-[6-methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine) in substantial stereochemical purity, comprising the steps of
a). forming a mixture of diastereomeric salts of compound 11 ((+)-2-{(E)-2-[6-methoxy-5-(4-methyl-1H-imidazol-1-yl)pyridin-2-yl]vinyl}-8-[2-(trifluoromethyl)phenyl]-5,6,7,8-tetrahydro[1,2,4]triazolo[1,5-a]pyridine) and compound 12 by treating a mixture of compound 11 and compound 12 with a chiral carboxylic acid compound;
b). crystallizing the diastereomeric salt formed of compound 12 from a solution of diastereomeric salts formed of compound 11 and compound 12; and
c). forming compound 12 from the obtained diastereomeric salt of compound 12.

5. The process according to claim 1, wherein the chiral carboxylic acid compound is selected from D-dibenzoyl tartaric acid (D-DBTA), D-dipivaloyl tartaric acid (D-DPTA) and (+)-N-(1-Phenylethyl)phthalamic acid ((+)-PEPA).

6. The process according to claim 1, wherein the solvent is a co-solvent mixture of 2-propanol and acetonitrile.

7. The process according to claim 1, wherein the solvent is a co-solvent mixture of methanol and acetonitrile.

8. The process according to claim 1 further comprising a second crystallization of the diastereomeric salt of compound 12 from a solvent prior to forming compound 12.

9. The process according to claim 8, wherein the solvent for the second crystallization is a co-solvent of 2-propanol and acetonitrile.

10. A D-DBTA salt of Compound 12.

11. A D-DPTA salt of Compound 12.

12. A (+)-N-(1-Phenylethyl)phthalamic acid ((+)-PEPA) salt of Compound 12.

13. A compound of Formula I: 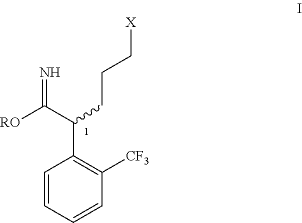
wherein X, R and the stereochemistry at carbon 1 are as defined in claim 1, or a salt thereof.

14. A compound of Formula III: 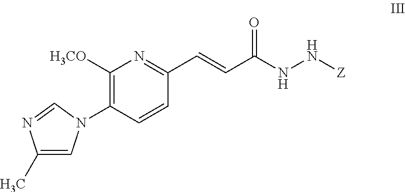
wherein Z is a hydrogen atom or a nitrogen protecting group, or a salt thereof.

15. The compound of Formula III or a salt thereof according to claim 14, wherein Z is a hydrogen atom.

16. A process for preparing a compound of Formula I, comprising the steps of
a). forming a compound of Formula VI by reacting 2-(trifluoromethyl)phenylacetonitrile with a compound of X(CH₂)₃X1 as shown below: 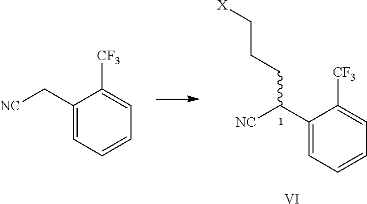
wherein X and X1 are leaving groups;
b). forming a compound of Formula I, by reacting a compound of Formula VI with ROH in the presence of an acid as shown below: 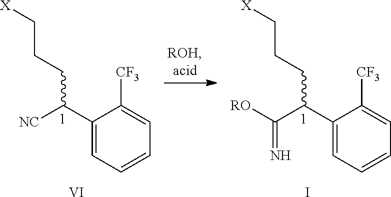
wherein X, R and the stereochemistry at carbon 1 are as defined in claim 1.

17. The process of claim 16, wherein the acid is in situ prepared by reacting a lower alkanoyl halide, thionyl chloride or trimethylsilyl halide with ROH.

18. A process for preparing a compound of Formula IV or a salt thereof, comprising the steps of
a). forming a compound of Formula III or a salt thereof by reacting N′-protected acrylohydrazide 5 or a salt thereof with a compound II or a salt thereof in the presence of palladium catalyst, a substituted phosphine of PR¹₃ and a base as shown below: 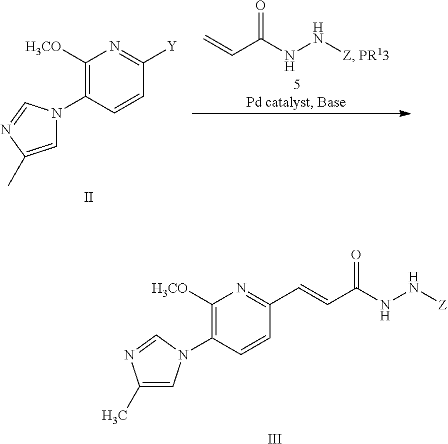
wherein Y is a leaving group; and R¹ is C1-C6 branched or unbranched alkyl group, or optionally substituted phenyl group;
b). forming a compound of Formula IV or a salt thereof by removing the protecting group of compound of Formula III as shown below:

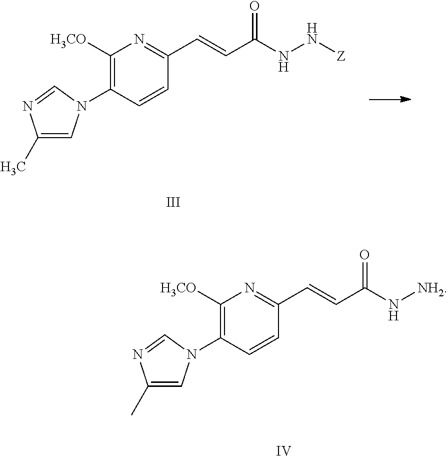

19. The process of claim 18, wherein dihydrochloride salt of compound of Formula IV is fouled by reacting a compound of Formula III with HCl in 1-propanol.

20. A compound of Formula II: 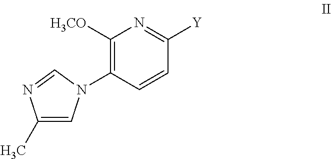
wherein Y is as defined in claim 18, or a salt thereof.

21. The compound according to claim 20, wherein Y is a bromine atom.
